(19) United States
(12) Patent Application Publication
(10) Pub. No.: US 2016/0074401 A1
(43) Pub. Date: Mar. 17, 2016

(54) COMPOSITIONS AND METHODS UTILIZING PHOSPHODIESTERASE INHIBITORS TO TREAT BLAST-INDUCED TINNITUS AND/OR HEARING LOSS

(71) Applicants: Jinsheng ZHANG, Troy, MI (US); Gulrez MAHMOOD, Plymouth, MI (US)

(72) Inventors: Jinsheng Zhang, Troy, MI (US); Gulrez Mahmood, Plymouth, MI (US)

(73) Assignee: WAYNE STATE UNIVERSITY, Detroit, MI (US)

(21) Appl. No.: 14/784,927

(22) PCT Filed: Apr. 17, 2014
(86) PCT No.: PCT/US14/34569

§ 371 Date: Oct. 15, 2015

Related U.S. Application Data
(60) Provisional application No. 61/813,524, filed on Apr. 18, 2013.

(51) Int. Cl. A61K 31/519

(52) U.S. Cl. CPC A61K 31/519

(57) ABSTRACT

The present disclosure provides compositions and methods utilizing phosphodiesterase inhibitors to treat blast-induced tinnitus and/or hearing loss. The compositions include phosphodiesterase inhibitors such as sildenafil.

### CROSS REFERENCE TO RELATED APPLICATIONS

This application claims the benefit under 35 U.S.C. §119(e) to U.S. Provisional Patent Application No. 61/813,524 filed Apr. 18, 2013, the entire contents of which are incorporated by reference herein.

### FIELD OF THE DISCLOSURE

The present disclosure describes compositions and methods utilizing phosphodiesterase inhibitors to treat blast-induced tinnitus and/or hearing loss.

### BACKGROUND OF THE DISCLOSURE

Tinnitus, often described as “ringing” in the ears, is the perception of sound that occurs in the absence of sound stimulus. Although the pathophysiology behind tinnitus remains elusive, it has been suggested that tinnitus occurs due to maladaptive plasticity changes following intense sound exposure.

Since the onset of the global War on Terror in 2001, blast-related traumatic brain injury (TBI) has become the signature injury among returning veterans. Auditory dysfunction is the most common sequela of blast-related injuries sustained on the battlefield. Over 62% of blast-injured veterans of Operation Iraqi Freedom (OIF) showed signs of hearing loss and over 38% exhibited tinnitus symptoms compared to only 15% in the general population (Heller, A., J Otolaryngol Clin North Am 36(2): 239-248, 2003). A related study revealed that over 53% of patients recovering from TBI exhibited tinnitus symptoms (Jury M A, Flynn M C., N Z Med J. 114(1134): 286-8, 2001). In fact, the most common service-connected disability payment veterans received in 2011 was for tinnitus (Administration, V. B., “Annual Benefits Report,” U.S. Department of Veterans Affairs, 2011). Thus, there appears to be a strong correlation between the plasticity changes that take place in TBI and the subsequent development of tinnitus and hearing loss (Lew, H. L. et al., J Rehabil Res Dev 44(7): 921-928, 2007).

Over 50 million Americans suffer from some form of tinnitus and over 30% of this population experiences severe debilitation. Despite the high prevalence of tinnitus, no definitive therapeutic intervention currently exists with the Cochrane Clinical Review finding incomplete or inconclusive evidence to endorse any particular treatment as appropriate for tinnitus.

### SUMMARY OF THE DISCLOSURE

This disclosure describes compositions and methods utilizing phosphodiesterase inhibitors (PDE-I(s)) to treat blast-induced tinnitus and/or hearing loss. The treatment can be by pharmacologic mitigation of a blast-induced traumatic brain injury (TBI).

### BRIEF DESCRIPTION OF THE FIGURES

FIG. 1 shows a diagram of a close-up of a pressure transducer and rat holder of a shock tube apparatus used for inducing blasts in experimental protocols described herein.

FIG. 2 shows surface righting latency results following each of the three blast or pseudo-blast exposures in Treated, Untreated and Sham groups.

FIGS. 3 and 4 show Gap-detection ratio values (Gap; Gap detection/startle only response) measured at 0-2 weeks (FIG. 3A), 2-4 weeks (FIG. 3B), 4-6 weeks (FIG. 4A), and 6-8 weeks (FIG. 4B) after blast exposure. Note that the Treated and Untreated groups showed significant differences in Gap-detection from 0-6 weeks post-blast (FIGS. 3A, 3B and 4A). Further recovery was demonstrated in both Treated and Untreated groups from 6-8 weeks (FIG. 4B). These indicate positive therapeutic effects of sildenafil. Error bars represent standard error of the mean (*p<0.05; BBN, broadband noise; [symbol] , post-blast Treated and Untreated groups Gap ratio returns to pre-blast levels; [symbol] , Only the post-blast Treated group Gap ratio returns to pre-blast levels).

FIGS. 5 and 6 show prepulse inhibition (PPI) ratio values (PPI/startle only response) measured at 0-2 weeks (FIG. 5A), 2-4 weeks (FIG. 5B), 4-6 weeks (FIG. 6A), and 6-8 weeks (FIG. 6B) after blast exposure. Note that the Treated and Untreated groups showed significant differences in PPI inhibition at all frequencies from 0-2 weeks post-blast, followed by marked recovery from 2-8 weeks post-blast, at most frequencies. This data indicates the acute therapeutic effect of PDE-I(s) including sildenafil on tinnitus, as well as a complex time course change. Error bars represent standard error of the mean (*p<0.05; BBN; [symbol] , post-blast Treated and Untreated groups Gap ratio returns to pre-blast levels; [symbol] , Only the post-blast Treated group Gap ratio returns to pre-blast levels).

FIG. 7 shows P1N1 amplitudes (wave 1) of the exposed ear at 28 kHz, comparing pre-blast (FIG. 7A) and post-blast (FIG. 7B) levels. Wave 1 amplitudes show an upward trend and are similar between Treated and Untreated groups. At 6 weeks post-blast, although there are no significant differences between the two groups at time-matched points, both Treated and Untreated groups show significant decline in wave 1 amplitudes, particularly at higher sound pressure levels (35+dB, SPL). For both Treated and Untreated groups, there is a significant difference in the P1N1 wave amplitude at 50 dB (p<0.01) and 45 dB (p<0.01).

FIGS. 8-12 show the percent change (from baseline) of Gap and PPI ratios for the Treated, Untreated, and Sham groups during post-blast weeks 1, 3, 4, 6, and 7. During post-blast week 1, both the Treated and Untreated groups showed significant upward percent change across all Gap ratio frequencies, indicating tinnitus (FIG. 8A). Both groups also showed significant upward change across all PPI ratio frequencies, indicating auditory detection deficits, however the Untreated group exhibited stronger deficits at several frequencies (FIG. 8B). By post-blast week 3, the Untreated group demonstrated tinnitus presence at 18-20 kHz and particularly robust tinnitus at 26-28 kHz (FIG. 9A), while the Treated group showed tinnitus suppression at 26-28 kHz, despite tinnitus presence at 6-8 kHz and 18-20 kHz. In contrast, although both groups displayed auditory detection deficits from 10-28 kHz (FIG. 9B), the Treated group showed a greater deficit at 14-16 kHz. At post-blast week 4, the Untreated group showed tinnitus presence from 14-28 kHz and BBN, while the Treated group only showed tinnitus at 18-20 kHz (FIG. 10A). Auditory detection deficits were seen from 10-20 kHz in the Untreated group at post-blast week 4, and from 10-28 kHz and BBN in the Treated group (FIG. 10B). At 6 weeks post-blast, the Untreated group exhibited tinnitus at 14-16 kHz and 26-28 kHz, while the Treated group showed tinnitus at 14-16 kHz and 18-20 kHz and suppression at 26-28 kHz (FIG. 11A). The Untreated group, however, showed auditory detection deficits at 18-20 kHz (FIG. 11B). The Treated group showed auditory detection deficits from 10-20 kHz. By the 7 week post-blast time point, the Untreated group retained 26-28 kHz tinnitus while 6-8 kHz and 26-28 kHz tinnitus reemerged in the Treated group (FIG. 12A). Both the Untreated and Treated groups displayed auditory detection deficits from 6-20 kHz, with the Treated group also showing deficits at 26-28 kHz and BBN (FIG. 12B).

FIGS. 13-17 show the percent change (from baseline) of startle force in response to the startle only condition with background noise (Gap-detection) and without PPI for the Treated, Untreated, and Sham groups during post-blast week 1, 3, 4, 6, and 7. During the first week post-blast, both Untreated and Treated groups showed significant startle force decrease in response to the startle only condition with (FIG. 13A) and without (FIG. 13B) background noise. By post-blast week 3, the Untreated group only showed a startle force decrease during 14-16 kHz background noise while the Treated group showed decrease during all frequencies (FIG. 14A). In the absence of background noise, the Untreated group showed little change except for an increase near 6-8 kHz and BBN prepulses, whereas the Treated group showed decreases near all frequencies (FIG. 14B). At post-blast week 4, the Untreated group demonstrated no startle force decreases during background noise (FIG. 15A) and increased startle force near 26-28 kHz and BBN prepulses (FIG. 15B), while the Treated group demonstrated decreases across all frequencies during and without background noise (FIGS. 15A and 15B). At 6 weeks post-blast, the Untreated group only showed decreased startle force during 14-16 kHz background noise (FIG. 16A), but showed significantly greater startle force than the Sham or Treated groups in the absence of background noise (FIG. 16B). The Treated group showed decreased startle force during all frequencies of background noise (FIG. 16A) but similar startle force to the Sham group in the absence of background noise (16B). Seven-week post-blast data revealed decreased startle force in the Untreated group from 10-28 kHz and in the Treated group during all background noise frequencies (FIG. 17A), as well as an increase in startle force near 26-28 kHz prepulses for the Untreated group and decreased startle force across all conditions for the Treated group (FIG. 17B).

FIG. 18 shows the ABR threshold shifts obtained during post-blast day 0, and post-blast weeks 1, 3, and 6 for the Treated, Untreated, and Sham groups in the exposed left ear (FIG. 18A) and plugged right ear (FIG. 18B). At post-blast day 0, significant threshold shifts averaging between 55-80 dB were observed in the exposed ear of the Treated and Untreated groups in clicks and tone bursts (FIG. 18A). The occluded ears with ear plugs still sustained significant threshold shifts across all tone burst frequencies, however, the Treated group demonstrated an overall decrease in frequency threshold shifts compared to the Untreated group (FIG. 18B). By post-blast week 1, the Treated group exhibited an overall decrease in frequency threshold shifts compared to the Untreated group in the exposed ear (FIG. 18A). While both groups still exhibited significant threshold shifts in the plugged ear (FIG. 18B), there were no longer significant differences between groups. From the third week post-blast onward, threshold shifts recovered for both groups in the occluded ear (FIG. 18B). In the exposed ear, significant threshold shifts were observed from 16-28 kHz during post-blast week 3 and 6 for the Untreated group, and in the Treated group at 8 kHz and 16-28 kHz during post-blast week 3 and at all frequencies during post-blast week 6 (FIG. 18A). There were, however, no significant differences between the Treated and Untreated groups at post-blast weeks 3 and 6.

### DETAILED DESCRIPTION

A number of therapeutic strategies have been tested as potential treatment measures against tinnitus and hearing loss. To date, however, there have been no sufficiently effective treatments.

The present disclosure describes compositions and methods utilizing phosphodiesterase inhibitors (PDE-I(s)) to treat blast-induced tinnitus and/or hearing loss. The treatment can be by pharmacologic mitigation of a blast-induced traumatic brain injury (TBI).

In recent years, a number of studies have investigated the neuroprotective potential of PDE-I(s) including PDE-5 inhibitors. For example, several studies on rats have reported positive therapeutic effects of PDE-5 inhibitors on the brain following an episode of ischemic stroke. Sildenafil treated rats exposed to embolic stroke exhibited improvement in angiogenesis, selective increase in cerebral blood flow, and enhanced neurological functional recovery up to 6 weeks post-injury. Treatment with a sildenafil-related PDE, Zaprinast, following focal cerebral cryolesion resulted in improved functional recovery, reduced oxidative stress, and prevention of apoptotic cell death. Furthermore, sildenafil treatment has also been shown to prevent hearing loss in noise-exposed guinea pigs. ABR threshold shifts were much smaller in the sildenafil treated group (4.8 dB) compared to the untreated group (22.0 dB) at 4 weeks post-noise exposure, suggesting a protective effect of sildenafil against noise induced hearing loss.

Nitric Oxide (NO), cyclic guanosine monophosphate (cGMP), and NO precursor L-arginine are key players in the signaling cascade and mediate the physiological function of PDE-I(s) like sildenafil. Accumulated intracellular cGMP permits increased vasodilation, reduced platelet aggregation, and reduced neutrophil adhesion in bovine intrapulmonary artery. Neurospheres isolated from the subventricular zone of adult rat brains show increased cGMP concentration when treated with sildenafil. The upregulation of cGMP was shown to enhance neurogenesis via activation of downstream effectors phosphatidyl inositol-3-kinase, Akt, and glycogen synthase kinase 3. Studies focusing on the cGMP/PKG/CREB pathway found that inhibitors of guanylate cyclase block the induction of long-term potentiation, whereas cGMP analogs promote potentiation.

The diverse physiological functions described share common signaling pathways that can be utilized to treat other neurological disorders following brain trauma. For example, increased cGMP triggers smooth muscle relaxation, vasodilation, and the downstream neuroprotective effects of NO expression in ischemic brain injury as well cryolesion-induced TBI. Administration of L-arginine, a precursor to NO, during the early phase of TBI (up to the first 15 minutes) results in increased blood flow, increased NO production, and enhanced recovery following TBI using the cortical impact model and the fluid-percussion injury model. NO is responsible for delaying rat neuronal sympathetic cell death following removal of neurotropic factors from the cell medium via activation of the cGMP-NO pathway. Furthermore, by adding cGMP analogs or cGMP PDE dipyridamole, neuronal loss is even further delayed. Thus, a growing body of evidence suggests that PDE-I(s), including PDE-5 inhibitors, and the related cGMP-NO biochemical pathway play a pivotal role in conveying cytoprotection to neuronal cells against a multitude of brain injury models.

A new model of NO synthesis following TBI has been proposed which describes an immediate initial spike in NO production within 5-30 minutes of exposure to TBI. Using transgenic eNOS−/− and nNOS−/−mice, it has been determined that 90% of the NO observed in this initial spike originates from neuronal nitric oxide synthase (nNOS) and the remaining 10% of the NO originates from endothelial nitric oxide synthase (eNOS). The initial spike of eNOS has been shown to be neuroprotective. Sildenafil has been shown to induce eNOS production in multiple cell types including human umbilical vein endothelial cells, cardiac myocytes, and retinal arterioles. Furthermore, sildenafil induced eNOS-phosphorylation conveys cytoprotection against ischemic myocytic injury and nerve crush injury by inhibition of apoptotic pathways. In the investigations described herein, it was attempted to not only augment eNOS production in the initial stages of acute blast exposure, but maintain eNOS upregulation until plasticity changes consolidated.

The current disclosure describes application of the cytoprotection of PDE-I(s) including sildenafil pharmacotherapy to treat blast-induced tinnitus and/or hearing loss. The compositions and methods were assessed in a model of blast-induced tinnitus, hearing loss, and related TBI. Animals were behaviorally tested to establish a pre-blast Gap baseline, then triple-blasted at 22 pounds per square inch (psi) with 15 minute intervals. The Treated group received 14 days total of PDE-I(s) starting at day 0 post-blast. Gap-detection and PPI behavioral testing were conducted for 8 weeks to assess long-term tinnitus symptomatology. ABR testing was performed at 1, 3, and 6 week post-blast time-points to assess hearing loss, and animals were euthanized at 8 weeks post-blast. The results described herein indicate a significant improvement in ABR hearing thresholds of the sildenafil Treated group at the 1 week time point; although the Untreated group eventually showed similar levels of hearing loss improvement after the 1 week time point. Furthermore, in the sildenafil Treated group, tinnitus perception is significantly reduced up to 6 weeks after blast-induced TBI.

As used herein, a “blast” is a pressure wave of a highly compressed medium, such as a gas or liquid. In one embodiment, a blast that induces tinnitus and/or hearing loss is a pressure wave measuring 10 psi or greater. In particular embodiments, the blast that induces tinnitus and/or hearing loss is a pressure wave measuring 11 psi or greater, 12 psi or greater, 13 psi or greater, 14 psi or greater, 15 psi or greater, 16 psi or greater, 17 psi or greater, 18 psi or greater, 19 psi or greater, 20 psi or greater, 21 psi or greater, 22 psi or greater, 23 psi or greater, 24 psi or greater, 25 psi or greater, 30 psi or greater, 35 psi or greater, 40 psi or greater, 50 psi or greater, and/or 100 psi or greater. In some embodiments, one or more blasts induce tinnitus and/or hearing loss including 1, 2, 3, 4 or 5 blasts.

PDE-I(s) disclosed for use with the compositions and methods disclosed herein include compounds represented by the Formula 1:

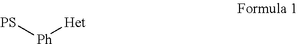

wherein Ph is optionally substituted phenylene; and PS is optionally substituted piperazinesulfonyl; and Het is optionally substituted pyrazolopyrimidinone.

With respect to any relevant structural representation, such as Formula 1, Ph can be an optionally substituted phenylene, such as optionally substituted m-phenylene. If Ph is substituted, it may have 1, 2, 3, or 4 substituents. Any substituent may be included on the phenylene. In some embodiments, some or all of the substituents on the phenylene may have: from 0-10 carbon atoms and from 0-10 heteroatoms, wherein each heteroatom is independently: O, N, S, F, Cl, Br, or I (provided that there is at least 1 non-hydrogen atom); and/or a molecular weight of 15-500 g/mol. For example, the substituents may be C₁₋₂₀ alkyl, such as CH₃, C₂H₅, C₃H₇, cyclic C₃H₅, C₄H₉, cyclic C₄H₇, C₅H₁₁, cyclic C₅H₉, C₆H₁₃, cyclic C₆H₁₁, etc.; C₁₋₂₀ alkoxyl; C₁₋₂₀ hydroxyalkyl; halo, such as F, Cl, Br, or I; OH; CN; NO₂; C₁₋₆ fluoroalkyl, such as CF₃, CF₂H, C₂F₅, etc.; a C₁₋₁₀ ester such as —O₂CCH₃, —CO₂CH₃, —O₂CC₂H₅, —CO₂C₂H₅, —O₂C-phenyl, —CO₂-phenyl, etc.; a C₁₋₁₀ ketone such as —COCH₃, —COC₂H₅, —COC₃H₇, —CO-phenyl, etc.; or a C₁₋₁₀ amine such as NH₂, NH(CH₃), N(CH₃)₂, N(CH₃)C₂H₅, etc. In some embodiments a substituent of Ph is C₁₋₁₂ alkyl or C₁₋₁₂—O-alkyl. In some embodiments, Ph is:

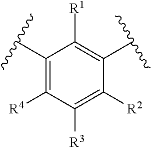

With respect to any relevant structural representation, such as Formula 1, PS is optionally substituted piperazinesulfonyl. If PS is substituted, it may have 1, 2, 3, 4, 5, 6, 7, 8, or 9 substituents. Any substituent may be included on the piperazinesulfonyl. In some embodiments, some or all of the substituents on the piperazinesulfonyl may have: from 0-10 carbon atoms and from 0-10 heteroatoms, wherein each heteroatom is independently: O, N, S, F, Cl, Br, or I (provided that there is at least 1 non-hydrogen atom); and/or a molecular weight of 15-500 g/mol. For example, the substituents may be C₁₋₂₀ alkyl, such as CH₃, C₂H₅, C₃H₇, cyclic C₃H₅, C₄H₉, cyclic C₄H₇, C₅H₁₁, cyclic C₅H₉, C₆H₁₃, cyclic C₆H₁₁, etc.; C₁₋₂₀ alkoxyl; C₁₋₂₀ hydroxyalkyl; halo, such as F, Cl, Br, or I; OH; CN; NO₂; C₁₋₆ fluoroalkyl, such as CF₃, CF₂H, C₂F₅, etc.; a C₁₋₁₀ ester such as —O₂CCH₃, —CO₂CH₃, —O₂CC₂H₅, —CO₂C₂H₅, —O₂C-phenyl, —CO₂-phenyl, etc.; a C₁₋₁₀ ketone such as —COCH₃, —COC₂H₅, —COC₃H₇, —CO-phenyl, etc.; or a C₁₋₁₀ amine such as NH₂, NH(CH₃), N(CH₃)₂, N(CH₃)C₂H₅, etc. In some embodiments a substituent of PS is C₁₋₁₂ alkyl or C₁₋₁₂—O-alkyl. In some embodiments, PS is:

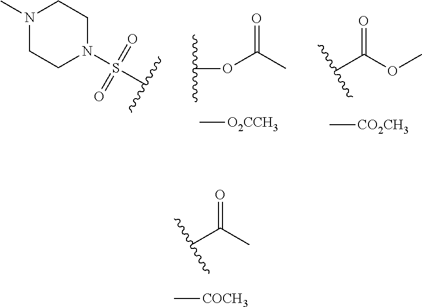

With respect to any relevant structural representation, such as Formula 1, Het is optionally substituted pyrazolopyrimidinonyl, such as optionally substituted pyrazolopyrimidinon-5-yl. If Het is substituted, it may have 1, 2, or 3 substituents. Any substituent may be included on the pyrazolopyrimidinonyl. In some embodiments, some or all of the substituents on the pyrazolopyrimidinonyl may have: from 0-10 carbon atoms and from 0-10 heteroatoms, wherein each heteroatom is independently: O, N, S, F, Cl, Br, or I (provided that there is at least 1 non-hydrogen atom); and/or a molecular weight of 15-500 g/mol. For example, the substituents may be C₁₋₂₀ alkyl, such as CH₃, C₂H₅, C₃H₇, cyclic C₃H₅, C₄H₉, cyclic C₄H₇, C₅H₁₁, cyclic C₅H₉, C₆H₁₃, cyclic C₆H₁₁, etc.; C₁₋₂₀ alkoxyl; C₁₋₂₀ hydroxyalkyl; halo, such as F, Cl, Br, or I; OH; CN; NO₂; C₁₋₆ fluoroalkyl, such as CF₃, CF₂H, C₂F₅, etc.; a C₁₋₁₀ ester such as —O₂CCH₃, —CO₂CH₃, —O₂CC₂H₅, —CO₂C₂H₅, —O₂C-phenyl, —CO₂-phenyl, etc.; a C₁₋₁₀ ketone such as —COCH₃, —COC₂H₅, —COC₃H₇, —CO-phenyl, etc.; or a C₁₋₁₀ amine such as NH₂, NH(CH₃), N(CH₃)₂, N(CH₃)C₂H₅, etc. In some embodiments a substituent of Het is C₁₋₁₂ alkyl or C₁₋₁₂—O-alkyl. In some embodiments, Het is:

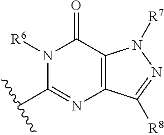

Some embodiments include a compound represented by Formula 2:

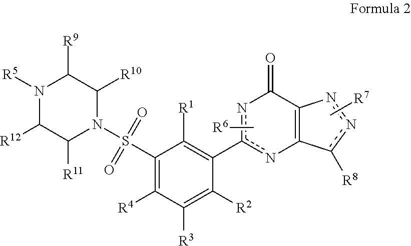

In Formula 2, the two adjacent dashed lines indicate a double bond in one position of the two dashed lines and a single bond in the other position. R⁶ and R⁷ are attached to the carbon or nitrogen atom that does not form the double bond. Formulas 3-8 are examples of possibilities that arise from the variable positions of the double bonds, R⁶, and R⁷.

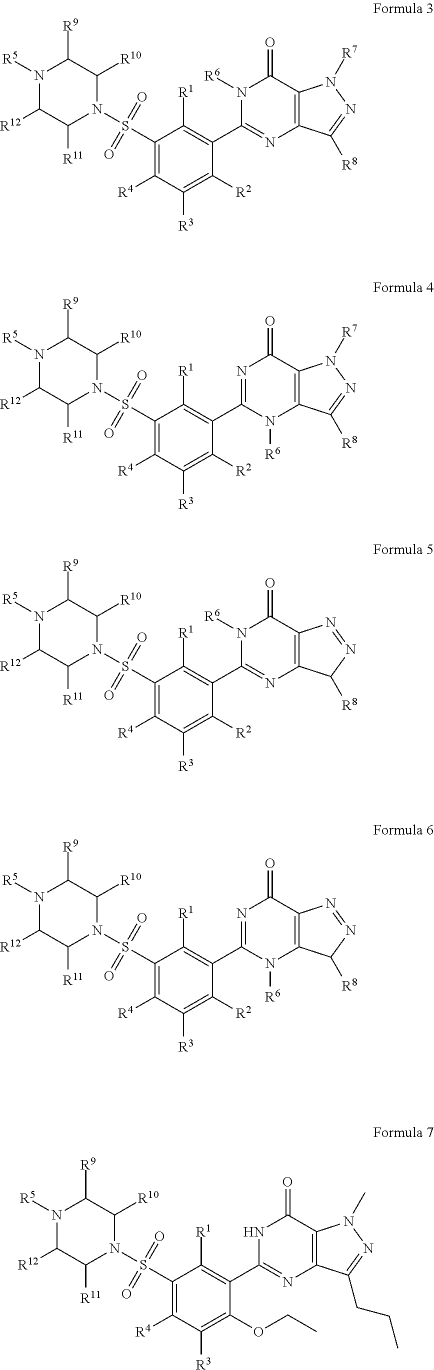

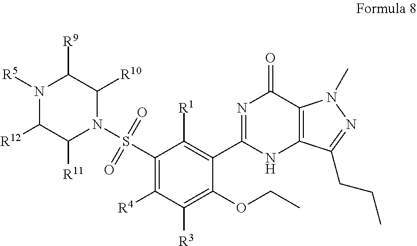

If stereochemistry is not indicated, such as in Formulas 1-8, a name or structural depiction includes any stereoisomer or any mixture of stereoisomers.

With respect to any relevant structural representation, such as Formulas 2-8; R¹, R², R³, R⁴, R⁵, R⁶, R⁷, R⁸, R⁹, R¹⁰, R¹¹, and R¹² may independently be H or any substituent, such as a substituent having from 0-6 carbon atoms and from 0-5 heteroatoms, wherein each heteroatom is independently: O, N, S, F, Cl, Br, or I; and/or having a molecular weight of 15-300 g/mol, or 15-150 g/mol. In some embodiments, R⁴, R⁵, R⁶, and R⁷ are independently R^{A}, F, Cl, CN, OR^{A}, CF₃, NO₂, NR^{A}R^{B}, COR^{A}, CO₂R^{A}, OCOR^{A}, NR^{A}COR^{B}, CONR^{A}R^{B}, etc. In some embodiments, R⁴, R⁵, R⁶, and R⁷ are independently H; F; Cl; CN; CF₃; OH; NH₂; C₁₋₆ alkyl, such as methyl, ethyl, propyl isomers (e.g. n-propyl and isopropyl), cyclopropyl, butyl isomers, cyclobutyl isomers (e.g. cyclobutyl and methylcyclopropyl), pentyl isomers, cyclopentyl isomers, hexyl isomers, cyclohexyl isomers, etc.; or C₁₋₆ alkoxy, such as —O-methyl, —O-ethyl, isomers of —O-propyl, —O-cyclopropyl, isomers of —O-butyl, isomers of —O-cyclobutyl, isomers of —O-pentyl, isomers of —O-cyclopentyl, isomers of —O-hexyl, isomers of —O-cyclohexyl, etc.

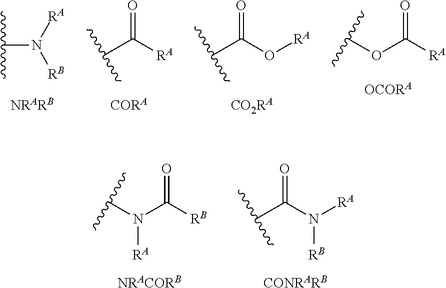

Each R^{A} may independently be H, or C₁₋₁₂ alkyl, including: linear or branched alkyl having a formula CₐHₐ₊₁, or cycloalkyl having a formula CₐHₐ₋₁, wherein a is 1, 2, 3, 4, 5, 6, 7, 8, 9, 10, 11, or 12, such as linear or branched alkyl of a formula: CH₃, C₂H₅, C₃H₇, C₄H₉, C₅H₁₁, C₆H₁₃, C₇H₁₅, C₈H₁₇, C₉H₁₉, C₁₀H₂₁, etc., or cycloalkyl of a formula: C₃H₅, C₄H₇, C₅H₉, C₆H₁₁, C₇H₁₃, C₅H₁₅, C₉H₁₇, C₁₀H₁₉, etc. In some embodiments, R^{A} may be H or C₁₋₆ alkyl. In some embodiments, R^{A} may be H or C₁₋₃ alkyl. In some embodiments, R^{A} may be H or CH₃. In some embodiments, R^{A} may be H.

Each R^{B} may independently be H, or C₁₋₁₂ alkyl, including: linear or branched alkyl having a formula CₐHₐ₊₁; or cycloalkyl having a formula CₐHₐ, wherein a is 1, 2, 3, 4, 5, 6, 7, 8, 9, 10, 11, or 12, such as linear or branched alkyl of a formula: CH₃, C₂H₅, C₃H₇, C₄H₉, C₅H₁₁, C₆H₁₃, C₅H₁₇, C₇H₁₅, C₉H₁₉, C₁₀H₂₁, etc., or cycloalkyl of a formula: C₃H₅, C₄H₇, C₅H₉, C₆H₁₁, C₇H₁₃, C₅H₁₅, C₉H₁₇, C₁₀H₁₉, etc. In some embodiments, R^{B} may be H or C₁₋₃ alkyl. In some embodiments, R^{B} may be H or CH₃. In some embodiments, such as R¹-R⁸, R^{B} may be H.

With respect to any relevant structural representation, such as Formulas 2-8, R¹ is H, or any substituent, such as a substituent having a molecular weight of 15-100 g/mol. In some embodiments, R¹ is NO₂, CN, H, F, Cl, Br, I, —CO₂H, —OH, C₁₋₆ alkylamino, C₁₋₆ alkyl, or C₁₋₆—O-alkyl. In some embodiments, R¹ is H. Additionally, for any embodiments above in this paragraph, R², R³, R⁴, R⁵, R⁶, R⁷, R⁸, R⁹, R¹⁰, R¹¹ and R¹², can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, or C₁₋₆ alkoxy. In some embodiments wherein R¹ is H; R², R³, and R⁴ can independently be H, C₁₋₄ alkyl, OH, C₁₋₄—O-alkyl, —CHO, C₂₋₄—CO-alkyl, C₂₋₄—CO-alkyl, CO₂H, C₂₋₄—CO₂-alkyl, F, Cl, Br, I, NO₂, or CN.

With respect to any relevant structural representation, such as Formulas 2-8, R² is H, or any substituent, such as a substituent having a molecular weight of 15-100 g/mol. In some embodiments, R² is NO₂, CN, H, F, Cl, Br, I, —CO₂H, —OH, C₁₋₆ alkylamino, C₁₋₆ alkyl, or C₁₋₆—O-alkyl. In some embodiments, R² is H. In some embodiments R² is —OCH₂CH₃. Additionally, for any embodiments above in this paragraph, R¹, R³, R⁴, R⁵, R⁶, R⁷, R⁸, R⁹, R¹⁰, R¹¹ and R¹², can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, or C₁₋₆ alkoxy. In some embodiments wherein R² is —OCH₂CH₃; R¹, R³, and R⁴ can independently be H, C₁₋₄ alkyl, OH, C₁₋₄—O-alkyl, —CHO, C₂₋₄—CO-alkyl, C₂₋₄—CO-alkyl, CO₂H, C₂₋₄—CO₂-alkyl, F, Cl, Br, I, NO₂, or CN.

With respect to any relevant structural representation, such as Formulas 2-8, R³ is H, or any substituent, such as a substituent having a molecular weight of 15-100 g/mol. In some embodiments, R³ is NO₂, CN, H, F, Cl, Br, I, —CO₂H, —OH, C₁₋₆ alkylamino, C₁₋₆ alkyl, or C₁₋₆—O-alkyl. In some embodiments, R³ is H. Additionally, for any embodiments above in this paragraph, R¹, R², R⁴, R⁵, R⁶, R⁷, R⁸, R⁹, R¹⁰, R¹¹ and R¹², can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, or C₁₋₆ alkoxy. In some embodiments wherein R³ is H; R¹, R², and R⁴, can independently be H, C₁₋₄ alkyl, OH, C₁₋₄—O-alkyl, —CHO, C₂₋₄—CO-alkyl, C₂₋₄—CO-alkyl, CO₂H, C₂₋₄—CO₂-alkyl, F, Cl, Br, I, NO₂, or CN.

With respect to any relevant structural representation, such as Formulas 2-8, R⁴ is H, or any substituent, such as a substituent having a molecular weight of 15-100 g/mol. In some embodiments, R⁴ is NO₂, CN, H, F, Cl, Br, I, —CO₂H, —OH, C₁₋₆ alkylamino, C₁₋₆ alkyl, or C₁₋₆—O-alkyl. In some embodiments, R⁴ is H. In some embodiments, R⁴ is Cl. Additionally, for any embodiments above in this paragraph, R¹, R², R³, R⁵, R⁶, R⁷, R⁸, R⁹, R¹⁰, R¹¹ and R¹², can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, or C₁₋₆ alkoxy. In some embodiments wherein R⁴ is H; R¹, R², and R³, can independently be H, C₁₋₄ alkyl, OH, C₁₋₄—O-alkyl, —CHO, C₂₋₄—CO-alkyl, C₂₋₄—CO-alkyl, CO₂H, C₂₋₄—CO₂-alkyl, F, Cl, Br, I, NO₂, or CN.

With respect to any relevant structural representation, such as Formulas 2-8, R⁵ is H, or any substituent, such as a substituent having a molecular weight of 15-100 g/mol. In some embodiments, R⁵ is NO₂, CN, H, F, Cl, Br, I, —CO₂H, —OH, C₁₋₆ alkylamino, C₁₋₆ alkyl, or C₁₋₆—O-alkyl. In some embodiments, R⁵ is H. In some embodiments R⁵ is —CH₃. Additionally, for any embodiments above in this paragraph, R¹, R², R³, R⁴, R⁶, R⁷, R⁸, R⁹, R¹⁰, R¹¹ and R¹², can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, or C₁₋₆ alkoxy. In some embodiments wherein R⁵ is —CH₃; R¹, R², R³, and R⁴, can independently be H, C₁₋₄ alkyl, OH, C₁₋₄—O-alkyl, —CHO, C₂₋₄—CO-alkyl, C₂₋₄—CO-alkyl, CO₂H, C₂₋₄—CO₂-alkyl, F, Cl, Br, I, NO₂, or CN.

With respect to any relevant structural representation, such as Formulas 2-8, R⁶ is H, or any substituent, such as a substituent having a molecular weight of 15-100 g/mol. In some embodiments, R⁶ is NO₂, CN, H, F, Cl, Br, I, —CO₂H, —OH, C₁₋₆ alkylamino, C₁₋₆ alkyl, or C₁₋₆—O-alkyl. In some embodiments, R⁶ is H. In some embodiments, R⁶ is Cl. Additionally, for any embodiments above in this paragraph, R¹, R², R³, R⁴, R⁵, R⁷, R⁸, R⁹, R¹⁰, R¹¹ and R¹², can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, or C₁₋₆ alkoxy. In some embodiments wherein R⁶ is H; R⁷ and R⁸, can independently be H, C₁₋₄ alkyl, OH, C₁₋₄—O-alkyl, —CHO, C₂₋₄—CO-alkyl, C₂₋₄—CO-alkyl, CO₂H, C₂₋₄—CO₂-alkyl, F, Cl, Br, I, NO₂, or CN.

With respect to any relevant structural representation, such as Formulas 2-8, R⁷ is H, or any substituent, such as a substituent having a molecular weight of 15-100 g/mol. In some embodiments, R⁷ is NO₂, CN, H, F, Cl, Br, I, —CO₂H, —OH, C₁₋₆ alkylamino, C₁₋₆ alkyl, or C₁₋₆—O-alkyl. In some embodiments, R⁷ is H. In some embodiments R⁷ is —CH₃. Additionally, for any embodiments above in this paragraph, R¹, R², R³, R⁴, R⁵, R⁶, R⁸, R⁹, R¹⁰, R¹¹ and R¹², can independently be: R^{A}, F, Cl, CN, OR^{A}, CF₃, NO₂, NR^{A}R^{B}, COR^{A} CO₂R^{A}, OCOR^{A}, NR^{A}COR^{B}, or CONR^{A}R^{B}; or H, F, Cl, CN, CF₃, OH, NH₂, C₁₋₆ alkyl, or C₁₋₆ alkoxy. In some embodiments wherein R⁷ is —CH₃; R⁶ and R⁸ can independently be H, C₁₋₄ alkyl, OH, C₁₋₄—O-alkyl, —CHO, C₂₋₄—CO-alkyl, C₂₋₄—CO-alkyl, CO₂H, C₂₋₄—CO₂-alkyl, F, Cl, Br, I, NO₂, or CN.

With respect to any relevant structural representation, such as Formulas 2-8, R⁸ is H, or any substituent, such as a substituent having a molecular weight of 15-100 g/mol. In some embodiments, R⁸ is NO₂, CN, H, F, Cl, Br, I, —CO₂H, —OH, C₁₋₆ alkylamino, C₁₋₆ alkyl, or C₁₋₆—O-alkyl. In some embodiments, R⁸ is H. In some embodiments R⁵ is —CH₂CH₂CH₃. Additionally, for any embodiments above in this paragraph, R¹, R², R³, R⁴, R⁵, R⁶, R⁷, R⁹, R¹⁰, R¹¹ and R¹², can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, or C₁₋₆ alkoxy. In some embodiments wherein R⁸ is n-propyl; R⁹, R¹⁰, R¹¹, and R¹² can independently be H, C₁₋₄ alkyl, OH, C₁₋₄—O-alkyl, —CHO, C₂₋₄—CO-alkyl, C₂₋₄—CO-alkyl, CO₂H, C₂₋₄—CO₂-alkyl, F, Cl, Br, I, NO₂, or CN.

With respect to any relevant structural representation, such as Formulas 2-8, R⁹ is H, or any substituent, such as a substituent having a molecular weight of 15-g/mol. In some embodiments, R⁹ is NO₂, CN, H, F, Cl, Br, I, —CO₂H, —OH, C₁₋₆ alkylamino, C₁₋₆ alkyl, or C₁₋₆—O-alkyl. In some embodiments, R⁹ is H. Additionally, for any embodiments above in this paragraph, R¹, R², R³, R⁴, R⁵, R⁶, R⁷, R⁸, R¹⁰, R¹¹ and R¹², can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, or C₁₋₆ alkoxy. In some embodiments wherein R⁹ is H; R¹⁰, R¹¹, and R¹² can independently be H, C₁₋₄ alkyl, OH, C₁₋₄—O-alkyl, —CHO, C₂₋₄—CO-alkyl, C₂₋₄—CO-alkyl, CO₂H, C₂₋₄—CO₂-alkyl, F, Cl, Br, I, NO₂, or CN.

With respect to any relevant structural representation, such as Formulas 2-8, R¹⁰ is H, or any substituent, such as a substituent having a molecular weight of 15-100 g/mol. In some embodiments, R¹⁰ is NO₂, CN, H, F, Cl, Br, I, —CO₂H, —OH, C₁₋₆ alkylamino, C₁₋₆ alkyl, or C₁₋₆—O-alkyl. In some embodiments, R¹⁰ is H. Additionally, for any embodiments above in this paragraph, R¹, R², R³, R⁴, R⁵, R⁶, R⁷, R⁸, R⁹, R¹¹ and R¹², can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, or C₁₋₆ alkoxy. In some embodiments wherein R¹⁰ is H; R⁹, R¹¹, and R¹² can independently be H, C₁₋₄ alkyl, OH, C₁₋₄—O-alkyl, —CHO, C₂₋₄—CO-alkyl, C₂₋₄—CO-alkyl, CO₂H, C₂₋₄—CO₂-alkyl, F, Cl, Br, I, NO₂, or CN.

With respect to any relevant structural representation, such as Formulas 2-8, R¹¹ is H, or any substituent, such as a substituent having a molecular weight of 15-100 g/mol. In some embodiments, R¹¹ is NO₂, CN, H, F, Cl, Br, I, —CO₂H, —OH, C₁₋₆ alkylamino, C₁₋₆ alkyl, or C₁₋₆—O-alkyl. In some embodiments, R¹¹ is H. Additionally, for any embodiments above in this paragraph, R¹, R², R³, R⁴, R⁵, R⁶, R⁷, R⁸, R⁹, R¹⁰ and R¹², can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, or C₁₋₆ alkoxy. In some embodiments wherein R¹¹ is H; R⁹, R¹⁰, and R¹² can independently be H, C₁₋₄ alkyl, OH, C₁₋₄—O-alkyl, —CHO, C₂₋₄—CO-alkyl, C₂₋₄—CO-alkyl, CO₂H, C₂₋₄—CO₂-alkyl, F, Cl, Br, I, NO₂, or CN.

With respect to any relevant structural representation, such as Formulas 2-8, R¹² is H, or any substituent, such as a substituent having a molecular weight of 15-100 g/mol. In some embodiments, R¹² is NO₂, CN, H, F, Cl, Br, I, —CO₂H, —OH, C₁₋₆ alkylamino, C₁₋₆ alkyl, or C₁₋₆—O-alkyl. In some embodiments, R¹² is H. Additionally, for any embodiments above in this paragraph, R¹, R², R³, R⁴, R⁵, R⁶, R⁷, R⁸, R⁹, R¹⁰ and R¹¹, can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, or C₁₋₆ alkoxy. In some embodiments wherein R¹² is H; R⁹, R¹⁰, and R¹¹ can independently be H, C₁₋₄ alkyl, OH, C₁₋₄—O-alkyl, —CHO, C₂₋₄—CO-alkyl, C₂₋₄—CO-alkyl, CO₂H, C₂₋₄—CO₂-alkyl, F, Cl, Br, I, NO₂, or CN.

Some embodiments include optionally substituted 1-[4-ethoxy-3-(6,7-dihydro-1-methyl-7-oxo-3-propyl-1H-pyrazolo[4,3-d]pyrimidin-5-yl)phenylsulfonyl]-4-methylpiperazine or a tautomer of 1-[4-ethoxy-3-(6,7-dihydro-1-methyl-7-oxo-3-propyl-1H-pyrazolo[4,3-d]pyrimidin-5-yl)phenylsulfonyl]-4-methylpiperazine, that is optionally substituted.

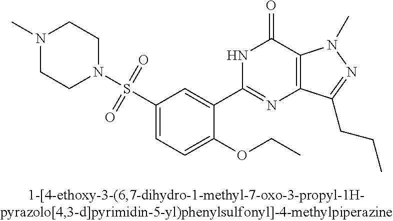 [SMILES: CCCc1nn(C)c2c(=O)[nH]c(-c3cc(S(=O)(=O)N4CCN(C)CC4)ccc3OCC)nc12]

The compound above can also be in its tautomeric form shown below, which would be considered equivalent to the structure above.

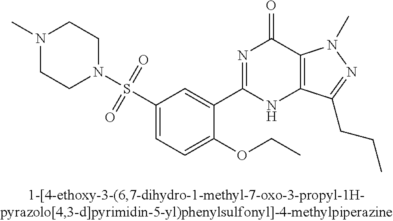 [SMILES: CCCc1nn(C)c2c(=O)nc(-c3cc(S(=O)(=O)N4CCN(C)CC4)ccc3OCC)[nH]c12]

Unless otherwise indicated, when a compound or chemical structural feature such as aryl is referred to as being “optionally substituted,” it includes a feature that has no substituents (i.e. unsubstituted), or a feature that is “substituted,” meaning that the feature has one or more substituents. The term “substituent” has the broadest meaning known to one of ordinary skill in the art, and includes a moiety that replaces one or more hydrogen atoms in a parent compound or structural feature. The term “replaces” is merely used herein for convenience, and does not require that the compound be formed by replacing one atom with another. In some embodiments, a substituent may be an ordinary organic moiety known in the art, which may have a molecular weight of 15-50 g/mol, 15-100 g/mol, 15-150 g/mol, 15-200 g/mol, 15-300 g/mol, or 15-500 g/mol. In some embodiments, a substituent includes: 0-30, 0-20, 0-10, or 0-5 carbon atoms; and 0-30, 0-20, 0-10, or 0-5 heteroatoms, wherein each heteroatom may independently be: N, O, S, Si, F, Cl, Br, or I; provided that the substituent includes one C, N, O, S, Si, F, Cl, Br, or I atom. A substituent should be sufficiently stable for a compound to be useful for the uses recited herein.

Examples of substituents include, but are not limited to, hydrocarbyl, such as alkyl, alkenyl, alkynyl; heteroalkyl, including any alkyl wherein one or more heteroatoms replaces: one or more carbon atoms and possibly some hydrogen atoms accompanying the carbon atoms (e.g. N replaces CH, O replaces CH₂, CI replaces CH₃, etc.), such as alkoxy, alkylthio, haloalkyl, haloalkoxy, amino, etc.; heteroalkenyl, including any alkenyl wherein one or more heteroatoms replaces: one or more carbon atoms and possibly some hydrogen atoms accompanying the carbon atoms, such as acyl, acyloxy, thiocarbonyl, alkylcarboxylate, O-carbamyl, N-carbamyl, O-thiocarbamyl, N-thiocarbamyl, C-amido, N-amido, sulfinyl, isocyanato, isothiocyanato, etc; heteroalkynyl, including any alkynyl wherein one or more heteroatoms replaces: one or more carbon atoms and possibly some hydrogen atoms accompanying the carbon atoms, such as cyano, thiocyanato, cyanato; aryl; heteroaryl; hydroxy; aryloxy; thiol; halo; S-sulfonamido; N-sulfonamido; nitro, silyl; sulfonyl; trihalomethanesulfonyl; trihalomethanesulfonamido; etc.

For convenience, the term “molecular weight” is used with respect to a moiety or part of a molecule to indicate the sum of the atomic masses of the atoms in the moiety or part of a molecule, even though it may not be a complete molecule.

The structures associated with some of the chemical names referred to herein are depicted below. These structures may be unsubstituted, as shown below, or a substituent may independently be in any position normally occupied by a hydrogen atom when the structure is unsubstituted. Unless a point of

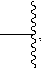

attachment is indicated by attachment may occur at any position normally occupied by a hydrogen atom.

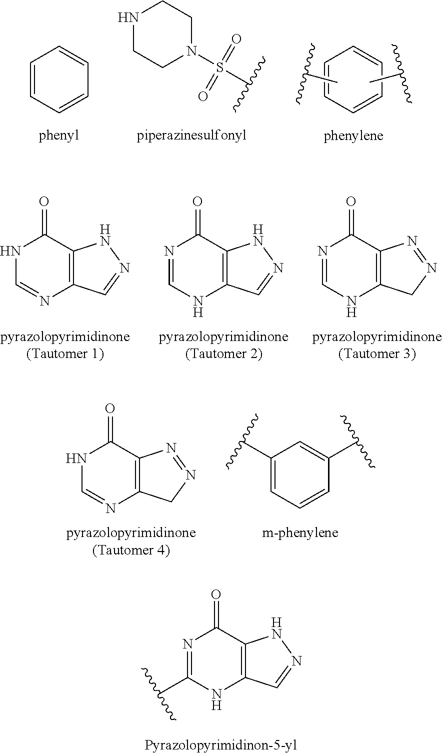

As shown above pyrazolopyrimidinone has at least 4 tautomeric forms.

As used herein, the term “alkyl” has the broadest meaning generally understood in the art, and may include a moiety composed of carbon and hydrogen containing no double or triple bonds. Alkyl may be linear alkyl, branched alkyl, cycloalkyl, or a combination thereof, and in some embodiments, may contain from 1-35 carbon atoms. In some embodiments, alkyl may include C₁₋₁₀ linear alkyl, such as methyl (—CH₃), ethyl (—CH₂CH₃), n-propyl (—CH₂CH₂CH₃), n-butyl (—CH₂CH₂CH₂CH₃), n-pentyl (—CH₂CH₂CH₂CH₂CH₃), n-hexyl (—CH₂CH₂CH₂CH₂CH₂CH₃), etc.; C₃₋₁₀ branched alkyl, such as C₃H₇ (e.g. iso-propyl), C₄H₉ (e.g. branched butyl isomers), C₅H₁₁ (e.g. branched pentyl isomers), C₆H₁₃ (e.g. branched hexyl isomers), C₇H₁₅ (e.g. heptyl isomers), etc.; C₃₋₁₀ cycloalkyl, such as C₃H₅ (e.g. cyclopropyl), C₄H₇ (e.g. cyclobutyl isomers such as cyclobutyl, methylcyclopropyl, etc.), C₅H₉ (e.g. cyclopentyl isomers such as cyclopentyl, methylcyclobutyl, dimethylcyclopropyl, etc.) C₆H₁₁ (e.g. cyclohexyl isomers), C₇H₁₃ (e.g. cycloheptyl isomers), etc.; and the like.

Particular embodiments disclosed herein utilize PDE-5 inhibitors. Exemplary PDE-5 inhibitors include sildenafil (1-[4-ethoxy-3-(6,7-dihydro-1-methyl-7-oxo-3-propyl-1H-pyrazolo[4,3-d]pyrimidin-5-yl)phenylsulfonyl]-4-methylpiperazine); tadalafil ((6R,12aR)-2,3,6,7,12,12a-Hexahydro-2-methyl-6-(3,4-methylene-dioxyphenyl) pyrazino(I′,2′:I,6)pyrido(3,4-b)indole-I,4-dione), vardenafil (2-(2-Ethoxy-5-(4-ethylpiperazin-I-yl-I-sulfonyl)phenyl)-5-methyl-7-propyl-3H-imidazo(5,1-f)(I,2,4 triazin-4-one); udenafil 5-[2-propyloxy-5-(I-methyl-2-pyrrolidinyl-ethyl-amidosulfonyl)phenyl]-methyl-3-propyl-I,6-dihydro-7H-pyrazolo(4,3-d)pyrimidine-7-one; dasantafil 7-(3-Bromo-4-methoxybenzyl)-I-ethyl-8-[[(I,2)-2-hydroxy cyclopentyl]amino]-3-(2-hydroxyethyl)-3,7-dihydro-1-purine-2,6-dione; avanafil 4-{[(3-chloro-4-methoxyphenyl)methyl]amino}-2-[(2S)-2-(hydroxymethyl)pyrrolidin-I-yl]-N-(pyrimidin-2-ylmethyl)pyrimidine-5-carboxamide; SLx 2101 of Surface Logix, LAS 3477PTriazolo[I,2-]xanthine,6-methyl-4-propyl-2-[2-propoxy-5-(4-methylpiperazino)-sulfonyl]phenyl or salts, hydrates or hydrates of salts thereof.

Additional PDE-I(s) including PDE-5 inhibitors that can be used in accordance with the present disclosure can be identified by assays that employ cells which express PDE (cell-based assays) or in assays with isolated PDE (cell-free assays). The various assays can employ a variety of variants of PDE-I(s) (e.g., full-length PDE-I(s), biologically active fragments of PDE-I(s), or fusion proteins, which include all or a portion of PDE-I(s)). The assays can be binding assays entailing direct or indirect measurement of the binding of a test compound or a known PDE ligand. The assays can also be activity assays entailing direct or indirect measurement of the activity of the PDE.

Some assays involve contacting PDE-5 with a test compound and determining the ability of the test compound to act as an antagonist of the enzymatic activity of PDE-5. These assays can monitor the PDE activity of PDE-5 by measuring the conversion of either cP or cGMP to its nucleoside monophosphate. The assays can also be expression assays entailing direct or indirect measurement of the expression of PDE-5 mRNA and PDE-5 protein. The various screening assays can be combined with an in vivo assay entailing measuring the effect of the test compound on blast-induced tinnitus and/or hearing loss.

Pharmaceutical compositions can be formed by combining PDE-I(s) disclosed herein, or pharmaceutically acceptable prodrugs or salts thereof, with a pharmaceutically acceptable carrier suitable for delivery to a subject in accordance with known methods of drug delivery and in particular dosages and amounts to achieve the beneficial effects disclosed herein. PDE-I(s) can also be provided as alternate solid forms, such as polymorphs, solvates, hydrates, etc.; tautomers; or any other chemical species that may rapidly convert to a PDE-I described herein under conditions in which the PDE-I(s) are used as described herein.

As used herein, a “pharmaceutically acceptable salt” refers to pharmaceutical salts that are, within the scope of sound medical judgment, suitable for use in contact with the tissues of subjects without undue toxicity, irritation, and allergic response, and are commensurate with a reasonable benefit/risk ratio. Pharmaceutically acceptable salts are well known in the art. In one embodiment, the pharmaceutically acceptable salt is a sulfate salt. For example, Berge, S. M. et al. describes pharmaceutically acceptable salts in J Pharm Sci 66:1-19, 1977.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids, examples of which are formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, and galacturonic acid. Pharmaceutically acceptable acidic/anionic salts also include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphospate, polygalacturonate, salicylate, stearate, subacetate, succinate, sulfate, hydrogensulfate, tannate, tartrate, teoclate, tosylate, and triethiodide salts.

Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine. All of these salts can be prepared by conventional means from the corresponding PDE-I represented by the disclosed PDE-I(s) by treating, for example, the disclosed PDE-I(s) with the appropriate acid or base. Pharmaceutically acceptable basic/cationic salts also include, the diethanolamine, ammonium, ethanolamine, piperazine and triethanolamine salts.

A pharmaceutically acceptable salt includes any salt that retains the activity of the parent PDE-I and is acceptable for pharmaceutical use. A pharmaceutically acceptable salt also refers to any salt which may form in vivo as a result of administration of an acid, another salt, or a prodrug which is converted into an acid or salt.

A prodrug includes a PDE-I which is converted to a therapeutically active PDE-I after administration, such as by hydrolysis of one or more functional groups or some other biologically labile group.

The PDE-I(s) disclosed herein can be provided as part of pharmaceutical compositions that include PDE-I(s) disclosed herein and at least one pharmaceutically acceptable excipient.

In some embodiments, the PDE-I(s) are provided as part of a composition that can include, for example, at least 0.1% w/v of PDE-I(s); at least 1% w/v of PDE-I(s); at least 10% w/v of PDE-I(s); at least 20% w/v of PDE-I(s); at least 30% w/v of PDE-I(s); at least 40% w/v of PDE-I(s); at least 50% w/v of PDE-I(s); at least 60% w/v of PDE-I(s); at least 70% w/v of PDE-I(s); at least 80% w/v of PDE-I(s); at least 90% w/v of PDE-I(s); at least 95% w/v of PDE-I(s); or at least 99% w/v of PDE-I(s).

In other embodiments, the PDE-I(s) are provided as part of a composition that can include, for example, at least 0.1% w/w of PDE-I(s); at least 1% w/w of PDE-I(s); at least 10% w/w of PDE-I(s); at least 20% w/w of PDE-I(s); at least 30% w/w of PDE-I(s); at least 40% w/w of PDE-I(s); at least 50% w/w of PDE-I(s); at least 60% w/w of PDE-I(s); at least 70% w/w of PDE-I(s); at least 80% w/w of PDE-I(s); at least 90% w/w of PDE-I(s); at least 95% w/w of PDE-I(s); or at least 99% w/w of PDE-I(s).

The PDE-I(s) and pharmaceutical compositions disclosed herein can be formulated for administration by, without limitation, injection, inhalation, ingestion, topical and/or transdermal application. The compositions disclosed herein can further be formulated for, without limitation, aural, intravenous, intradermal, intraarterial, intraperitoneal, topical, intrathecal, intramuscular, oral, subcutaneous, and/or transdermal administration.

For injection, compositions can be formulated as aqueous solutions, such as in buffers including Hanks' solution, Ringer's solution, or physiological saline. The aqueous solutions can contain formulatory agents such as suspending, stabilizing and/or dispersing agents. Alternatively, the formulation can be in lyophilized and/or powder form for constitution with a suitable vehicle, e.g., sterile pyrogen-free water, before use.

For oral administration, the compositions can be formulated as tablets, pills, dragees, capsules, liquids, gels, syrups, slurries, suspensions and the like. For oral solid formulations such as, for example, powders, capsules and tablets, suitable excipients include binders (gum tragacanth, acacia, cornstarch, gelatin), fillers such as sugars, e.g. lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

For aural administration, compositions can be formulated as ear drops, ointments, creams, liquids, gels, salves or powders for application to the ear, either superficially or internally. Aural formulations can be delivered via the external ear, middle ear and/or inner ear.

Aural formulations to the external ear can be modified based on pH, viscosity and tonicity. External ear formulations can include a physiologic pH ranging from 3.5 to 7.5; and solvents such as propylene glycol, glycerin, oils, and polymers can be added to increase viscosity and increase PDE-I(s) residence time in the ear canal by preventing the composition from running out of the ear canal. In addition, external ear formulations may include isotonic solutions to reduce irritation after application. Preservatives may also be added to reduce microbial growth; and ointment and powder preparations can be used to provide relatively higher drug retention times in the external ear.

Aural formulations for delivery to the middle ear and inner ear can be achieved by injection through the tympanic membrane into the middle ear, or using catheters or wicks for delivery through the tympanic membrane. To decrease drainage of the drug through the eustachian tube, the viscosity of liquid formulations can be increased using solvents such as glycerin and propylene glycol; or polymers such as sodium hyaluronate, gelatin, polypropylene fumarate, or biodegradable polymer gels. Dose device pumps and catheters can be used for manually or electronically controlled administration to the middle or inner ear. In addition, these aural formulations can include active PDE-I(s) that do not require chemical modifications, to avoid the need for metabolizing the drug to produce an active form.

Compositions including PDE-I(s) disclosed herein can be administered as an aerosol. In one embodiment, the aerosol delivery vehicle is an anhydrous, liquid or dry powder inhaler. PDE-I(s) can be included in a pharmaceutical composition formulated for delivery as a dry powder or aerosol for nasal, sinunasal or pulmonary administration.

For administration by inhalation, PDE-I(s) can be formulated as aerosol sprays from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator may be formulated containing a powder mix of the compound and a suitable powder base such as lactose or starch.

For transdermal administration, a patch can be used. Exemplary patches can have a skin contacting portion made of any suitable material that is covered or impregnated with PDE-I(s) described herein, wherein the skin contacting portion can be supported by a backing, one or both of which may have an adhesive segment or other configuration for attaching to a skin surface. Penetration enhancers, adjuvants, surfactants, lubricants, etc. can also be present in transdermal patches. Exemplary transdermal penetration enhancers include 1,3-dimethyl-2-imidazolidinone or 1,2-propanediol. Other examples include cationic, anionic, or nonionic surfactants (e.g., sodium dodecyl sulfate, polyoxamers, etc.); fatty acids and alcohols (e.g., ethanol, oleic acid, lauric acid, liposomes, etc.); anticholinergic agents (e.g., benzilonium bromide, oxyphenonium bromide); alkanones (e.g., n-heptane); amides (e.g., urea, N,N-dimethyl-m-toluamide); organic acids (e.g., citric acid); sulfoxides (e.g., dimethylsulfoxide); terpenes (e.g., cyclohexene); ureas; sugars; carbohydrates or other agents. Transdermal penetration enhancers can be present in any suitable amount.

Any composition formulation disclosed herein can advantageously include any other pharmaceutically acceptable carriers which include those that do not produce significantly adverse, allergic or other untoward reactions that outweigh the benefit of administration, whether for research, prophylactic and/or therapeutic treatments. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety and purity standards as required by US FDA Office of Biological Standards and/or other relevant foreign regulatory agencies.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, chelating agents (e.g., EDTA), gels, binders, disintegration agents, and/or lubricants.

Exemplary buffering agents include citrate buffers, succinate buffers, tartrate buffers, fumarate buffers, gluconate buffers, oxalate buffers, lactate buffers, acetate buffers, phosphate buffers, histidine buffers and/or trimethylamine salts.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol and 3-pentanol.

Exemplary isotonic agents include polyhydric sugar alcohols including trihydric or higher sugar alcohols, such as glycerin, erythritol, arabitol, xylitol, sorbitol or mannitol.

Exemplary stabilizers include organic sugars, polyhydric sugar alcohols, polyethylene glycol; sulfur-containing reducing agents, amino acids, low molecular weight polypeptides, proteins, immunoglobulins, hydrophilic polymers or polysaccharides.

Methods disclosed herein include treating subjects (humans, veterinary animals, livestock and research animals) with PDE-I(s) disclosed herein including salts and prodrugs thereof. Treating subjects can include delivering an effective amount and/or delivering a prophylactic treatment and/or a therapeutic treatment. An “effective amount” is the amount of PDE-I(s) necessary to result in a desired physiological change in the subject. Effective amounts are often administered for research purposes. Effective amounts disclosed herein can treat blast-induced tinnitus and/or hearing loss in animal models disclosed herein.

A “prophylactic treatment” includes a treatment administered to a subject who does not display signs or symptoms of blast-induced tinnitus and/or hearing loss or displays only early signs or symptoms of blast-induced tinnitus and/or hearing loss such that treatment is administered for the purpose of diminishing, preventing, or decreasing the risk of developing blast-induced tinnitus and/or hearing loss further. Thus, a prophylactic treatment functions as a preventative treatment against blast-induced tinnitus and/or hearing loss.

A “therapeutic treatment” includes a treatment administered to a subject who displays symptoms or signs of blast-induced tinnitus and/or hearing loss and is administered to the subject for the purpose of diminishing or eliminating those signs or symptoms of blast-induced tinnitus and/or hearing loss. The therapeutic treatment can reduce, control, or eliminate the presence of blast-induced tinnitus and/or hearing loss.

“Therapeutically effective amounts” can include those that provide effective amounts, prophylactic treatments and/or therapeutic treatments. In particular embodiments, therapeutically effective amounts provide effective amounts and/or amounts that provide prophylactic treatments. Therapeutically effective amounts need not fully prevent or cure blast-induced tinnitus and/or hearing loss but can also provide a partial benefit, such as reduction of blast-induced tinnitus and/or hearing loss. Therapeutically effective amounts can suppress blast-induced tinnitus 3-6 weeks following exposure to a blast. Therapeutically effective amounts can also suppress high frequency blast-induced tinnitus in the 26-28 kHz range. Therapeutically effective amounts can also suppress high frequency blast-induced tinnitus in the 26-28 kHz range 3-6 weeks following exposure to a blast. Therapeutically effective amounts can also reduce 6-8 kHz hearing loss; 14-16 kHz hearing loss and/or 18-20 kHz hearing loss following exposure to the blast

Therapeutically effective amounts that reduce, control, or eliminate the presence of blast-induced tinnitus and/or hearing loss can be measured by Gap-detection, PPI performance, and/or ABR as described herein. Exemplary methods to demonstrate therapeutically effective amounts in human subjects can include changed performances in ABR, audiogram, and distortion otoacoustic emissions that are commonly used clinically. See for example Rhodes, et al. Otolaryngol Head Neck Surg 120(6): 799-808, 1999 and Tsui, et al. Clin Otolaryngol 33(2): 108-112, 2008. Changed performance can be measured as a percentage change from a previous testing in the same subject or as a percentage difference from a control population or reference score. The percentage change can be 2.5%; 5%; 10%; 15%; 20%; 25%; 30%; 35%; 40%; 45%; 50%; 55%; 60%; 65%; 70%; 75%; 80%; 85%; 90%; 95%; 100% or more. The changed performance can also be lack of tinnitus within a frequency range and/or loss of previously-existing tinnitus within a frequency range. The changed performance can also be maintenance of auditory detection within a frequency range or the re-gaining of previously-lost auditory detection within a frequency range. The frequency range can include any frequency range disclosed herein.

For administration, therapeutically effective amounts (also referred to herein as doses) can be initially estimated based on results from in vitro assays and/or animal model studies. For example, a dose can be formulated in animal models to achieve improvements in Gap-detection, PPI performance, and/or ABR. Such information can be used to more accurately determine useful doses in subjects of interest.

The actual dose amount administered to a particular subject can be determined by a physician, veterinarian or researcher taking into account parameters such as physical and physiological factors including target, body weight, severity of condition, type of blast exposure, previous or concurrent therapeutic interventions, idiopathy of the subject and route of administration. In particular embodiments, the actual dose administered can be selected by a subject following exposure to a blast. In these embodiments, the subject will have received previous instructions regarding use of the PDE-I(s) in treating blast-induced tinnitus and/or hearing loss prior to blast exposure for which the treatment is administered.

The amount and concentration of PDE-I(s) in a pharmaceutical composition, as well as the quantity of the pharmaceutical composition administered to a subject, can be selected based on clinically relevant factors, the solubility of the PDE-I(s) in the pharmaceutical composition, the potency and activity of the PDE-I(s), and the manner of administration of the PDE-I(s).

Useful doses can often range from 0.1 μg/kg to 5 μg/kg and/or from 0.5 μg/kg to 1 μg/kg. In other non-limiting examples, a dose can include 1 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1000 μg/kg, 0.1 mg/kg to 5 mg/kg and/or from 0.5 mg/kg to 1 mg/kg. In other non-limiting examples, a dose can include 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 16 mg/kg, 17 mg/kg, 18 mg/kg, 19 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 150 mg/kg, 200 mg/kg, 250 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, 1000 mg/kg or more. In other non-limiting examples, a dose can include 1 mg/kg to 3 mg/kg, 2.5 mg/kg to 4.5 mg/kg, 4 mg/kg to 6 mg/kg, 5.5 mg/kg to 7.5 mg/kg, 7 mg/kg to 9 mg/kg, 8.5 mg/kg to 10.5 mg/kg, 10 mg/kg to 12 mg/kg, 11.5 mg/kg to 13.5 mg/kg, 13 mg/kg to 15 mg/kg, 14.5 mg/kg to 16.5 mg/kg, 16 mg/kg to 18 mg/kg, 17.5 mg/kg 19.5 mg/kg, 19 mg/kg to 21 mg/kg, 20.5 mg/kg to 22.5 mg/kg, 22 mg/kg to 24 mg/kg, 23.5 mg/kg to 25.5 mg/kg, and/or 25 mg/kg to 27 mg/kg.

In particular embodiments, the PDE-I(s) treatment of blast-induced tinnitus and/or hearing loss can be measured by Gap-detection, PPI performance, and/or ABR. In some embodiments, the PDE-I(s) treats blast-induced tinnitus and/or hearing loss for all frequencies, for only one subset of frequencies, or for one or more subsets of frequencies. In some embodiments, subsets of frequencies include frequencies of various ranges, low frequencies (6-12 kHz), middle frequencies (14-20 kHz) or high frequencies (26-28 kHz). In some embodiments, the PDE-I(s) treat blast-induced tinnitus and/or hearing loss for frequencies of 8 kHz, 12 kHz, 16 kHz, 20 kHz, 28 kHz, and/or for BBN. In some embodiments, the PDE-I(s) treat blast-induced tinnitus and/or hearing loss for frequencies of 8-12 kHz, 8-16 kHz, 8-20 kHz, 8-28 kHz, 12-16 kHz, 12-20 kHz, 12-28 kHz, 16-20 kHz, 16-28 kHz, and/or 20-28 kHz, In some embodiments, the PDE-I(s) treat the blast-induced tinnitus and/or hearing loss for frequencies of 6-8 kHz, 10-12 kHz, 14-16 kHz, 18-20 kHz, and/or 26-28 kHz. In some embodiments, the PDE-I(s) treat blast-induced tinnitus and/or hearing loss for frequencies of 6-12 kHz, 6-16 kHz, 6-28 kHz, 8-20 kHz, and 18-20 kHz. The PDE-I(s) can treat blast-induced tinnitus and/or hearing loss by producing a protective effect, or without producing a protective effect against TBI.

Therapeutically effective amounts can be achieved by administering single or multiple doses. In particular embodiments, the PDE-I(s) is administered within 24 hours; within 20 hours; within 16 hours; within 12 hours; within 6 hours; within 5 hours; within 4 hours; within 3 hours; within 2 hours; within 1 hour; within 30 minutes; within 29 minutes; within 28 minutes; within 27 minutes; within 26 minutes; within 25 minutes; within 24 minutes; within 23 minutes; within 22 minutes; within 21 minutes; within 20 minutes; within 19 minutes; within 18 minutes; within 17 minutes; within 16 minutes; within 15 minutes; within 14 minutes; within 13 minutes; within 12 minutes; within 11 minutes; within 10 minutes; within 9 minutes; within 8 minutes; within 7 minutes; within 6 minutes; within 5 minutes; within 4 minutes; within 3 minutes; within 2 minutes; within 1 minute; within 30 seconds; within 20 seconds; within 10 seconds or within 5 seconds of blast exposure. In particular embodiments, the PDE-I(s) can be administered for a course of 1 day, 2 days, 3 days, 4 days, 5 days, 6 days, 7 days, 8 days, 9 days, 10 days, 11 days, 12 days, 13 days, 14 days, 15 days, 16 days, 20 days, 21 days, 22 days, 23 days, 24 days, 25 days, 26 days, 27 days, 28 days, 29 days, 30 days, or more following blast exposure. The PDE-I(s) can be administered continuously or intermittently, for example, the PDE-I(s) can be administered for a number of days, followed by a number of days without PDE-I(s) administration, and then administration of PDE-I(s) for another number of days. The number of days can be the same or different and can be any number of days between 1-60.

In additional embodiments, the PDE-I can be administered to a subject who may be exposed to a blast before entering the situation where a blast may occur. In these embodiments, the PDE-I can be administered 5 seconds before; 10 seconds before; 20 seconds before; 30 seconds before; 1 minute before; 2 minutes before; 3 minutes before; 4 minutes before; 5 minutes before; 6 minutes before; 7 minutes before; 8 minutes before; 9 minutes before; 10 minutes before; 11 minutes before; 12 minutes before; 13 minutes before; 14 minutes before; 15 minutes before; 16 minutes before; 17 minutes before; 18 minutes before; 19 minutes before; 20 minutes before; 21 minutes before; 22 minutes before; 23 minutes before; 24 minutes before; 25 minutes before; 26 minutes before; 27 minutes before; 28 minutes before; 29 minutes before; 30 minutes before; 1 hour before; 2 hours before; 3 hours before; 4 hours before; 5 hours before; 6 hours before; 12 hours before; 16 hours before; 20 hours before or 24 hours before entering a situation where a blast may occur. In particular embodiments, the PDE-I(s) can be administered for a course of 1 day, 2 days, 3 days, 4 days, 5 days, 6 days, 7 days, 8 days, 9 days, 10 days, 11 days, 12 days, 13 days, 14 days, 15 days, 16 days, 20 days, 21 days, 22 days, 23 days, 24 days, 25 days, 26 days, 27 days, 28 days, 29 days, 30 days, or more before blast exposure.

In additional embodiments, the PDE-I(s) can be administered to a subject before exposure to a blast and after exposure to a blast. In additional embodiments, the PDE-I(s) can be administered before a subject enters a situation where a blast may occur and after exposure to a blast.

### Exemplary Embodiments

### Set 1

1. A method of treating blast-induced tinnitus in a subject in need thereof comprising administering to the subject a therapeutically effective amount of a phosphodiesterase inhibitor (PDE-I) within a time period associated with exposure to a blast thereby providing a therapeutic treatment by reducing blast-induced tinnitus in the subject following exposure to the blast.
2. A method of embodiment 1 where the PDE-I is a PDE-5 inhibitor.
3. A method of embodiment 1 or 2 wherein the PDE-I is sildenafil.
4. A method of any one of embodiments 1-3 wherein the time period is within 24 hours of exposure to a blast; within 1 hour of exposure to a blast; within 10 minutes of exposure to a blast; or within 5 minutes of exposure to a blast.
5. A method of any one of embodiments 1-4 wherein the blast creates a pressure wave of 10 psi or greater; 20 psi or greater; or 22 psi or greater.
6. A method of any one of embodiments 1-5 wherein the tinnitus is high-frequency tinnitus.
7. A method of any one of embodiments 1-6 wherein the therapeutic treatment reduces blast-induced tinnitus in the subject 0-6 weeks following exposure to the blast.
8. A method of any one of embodiments 1-7 wherein the therapeutic treatment reduces blast-induced tinnitus in the subject 0 weeks following exposure to the blast.
9. A method of any one of embodiments 1-8 wherein the therapeutic treatment reduces blast-induced tinnitus in the subject 1 week following exposure to the blast.
10. A method of any one of embodiments 1-9 wherein the therapeutic treatment reduces blast-induced tinnitus in the subject 2 weeks following exposure to the blast.
11. A method of any one of embodiments 1-10 wherein the therapeutic treatment reduces blast-induced tinnitus in the subject 3 weeks following exposure to the blast.
12. A method of any one of embodiments 1-11 wherein the therapeutic treatment reduces blast-induced tinnitus in the subject 4 weeks following exposure to the blast.
13. A method of any one of embodiments 1-12 wherein the therapeutic treatment reduces blast-induced tinnitus in the subject 5 weeks following exposure to the blast.
14. A method of any one of embodiments 1-13 wherein the therapeutic treatment reduces blast-induced tinnitus in the subject 6 weeks following exposure to the blast.
15. A method of any one of embodiments 1-14 wherein the therapeutic treatment reduces blast-induced tinnitus in the subject more than 6 weeks following exposure to the blast.
16. A method of any one of embodiments 1-8 wherein the therapeutic treatment reduces 26-28 kHz blast-induced tinnitus in the subject.
17. A method of any one of embodiments 1-16 wherein the therapeutic treatment reduces 6-8 kHz; 10-12 kHz; 14-16 kHz; 18-20 kHz; 22-24 kHz; and/or 26-28 kHz blast-induced tinnitus in the subject.
18. A method of any one of embodiments 1-17 wherein the therapeutic treatment reduces 6-8 kHz blast-induced tinnitus in the subject.
19. A method of any one of embodiments 1-18 wherein the therapeutic treatment reduces 10-12 kHz blast-induced tinnitus in the subject.
20. A method of any one of embodiments 1-19 wherein the therapeutic treatment reduces 14-16 kHz blast-induced tinnitus in the subject.
21. A method of any one of embodiments 1-20 wherein the therapeutic treatment reduces 18-20 kHz blast-induced tinnitus in the subject.
22. A method of any one of embodiments 1-21 wherein the therapeutic treatment reduces 22-24 kHz blast-induced tinnitus in the subject.
23. A method of any one of embodiments 1-22 wherein the therapeutic treatment reduces 26-28 kHz blast-induced tinnitus in the subject.
24. A method of treating hearing loss in a subject in need thereof comprising administering to the subject a therapeutically effective amount of a phosphodiesterase inhibitor (PDE-I) within a time period associated with exposure to a blast thereby providing a therapeutic treatment by reducing hearing loss in the subject.
25. A method of embodiment 24 where the PDE-I is a PDE-5 inhibitor.
26. A method of embodiment 24 or 25 wherein the PDE-I is sildenafil.
27. A method of any one of embodiments 24-26 wherein the time period is within 24 hours of exposure to a blast; within 1 hour of exposure to a blast; within 10 minutes of exposure to a blast; or within 5 minutes of exposure to a blast.
28. A method of any one of embodiments 24-27 wherein the blast creates a pressure wave of 10 psi or greater; 20 psi or greater; or 22 psi or greater.
29. A method of any one of embodiments 24-28 wherein the tinnitus is high-frequency tinnitus.
30. A method of any one of embodiments 24-29 wherein the therapeutic treatment reduces blast-induced tinnitus in the subject 0-6 weeks following exposure to the blast.
31. A method of any one of embodiments 24-30 wherein the therapeutic treatment reduces blast-induced tinnitus in the subject 0 weeks following exposure to the blast.
32. A method of any one of embodiments 24-31 wherein the therapeutic treatment reduces blast-induced tinnitus in the subject 1 week following exposure to the blast.
33. A method of any one of embodiments 24-32 wherein the therapeutic treatment reduces blast-induced tinnitus in the subject 2 weeks following exposure to the blast.
34. A method of any one of embodiments 24-33 wherein the therapeutic treatment reduces blast-induced tinnitus in the subject 3 weeks following exposure to the blast.
35. A method of any one of embodiments 24-33 wherein the therapeutic treatment reduces blast-induced tinnitus in the subject 4 weeks following exposure to the blast.
36. A method of any one of embodiments 24-35 wherein the therapeutic treatment reduces blast-induced tinnitus in the subject 5 weeks following exposure to the blast.
37. A method of any one of embodiments 24-36 wherein the therapeutic treatment reduces blast-induced tinnitus in the subject 6 weeks following exposure to the blast.
38. A method of any one of embodiments 24-37 wherein the therapeutic treatment reduces blast-induced tinnitus in the subject more than 6 weeks following exposure to the blast.
39. A method of any one of embodiments 24-38 wherein the therapeutic treatment reduces 26-28 kHz blast-induced tinnitus in the subject.
40. A method of any one of embodiments 24-39 wherein the therapeutic treatment reduces 6-8 kHz; 10-12 kHz; 14-16 kHz; 18-20 kHz; 22-24 kHz; and/or 26-28 kHz blast-induced tinnitus in the subject.
41. A method of any one of embodiments 24-40 wherein the therapeutic treatment reduces 6-8 kHz blast-induced tinnitus in the subject.
42. A method of any one of embodiments 24-41 wherein the therapeutic treatment reduces 10-12 kHz blast-induced tinnitus in the subject.
43. A method of any one of embodiments 24-42 wherein the therapeutic treatment reduces 14-16 kHz blast-induced tinnitus in the subject.
44. A method of any one of embodiments 24-43 wherein the therapeutic treatment reduces 18-20 kHz blast-induced tinnitus in the subject.
45. A method of any one of embodiments 24-44 wherein the therapeutic treatment reduces 22-24 kHz blast-induced tinnitus in the subject.
46. A method of any one of embodiments 24-45 wherein the therapeutic treatment reduces 26-28 kHz blast-induced tinnitus in the subject.

### Exemplary Embodiments

### Set 2

1. A method of treating blast-induced tinnitus and hearing loss in a subject in need thereof comprising administering to the subject a therapeutically effective amount of a phosphodiesterase inhibitor (PDE-I) within a time period associated with exposure to a blast thereby providing a therapeutic treatment by reducing blast-induced tinnitus and hearing in the subject following exposure to the blast.
2. A method of embodiment 1 where the PDE-I is a PDE-5 inhibitor.
3. A method of embodiment 1 or 2 wherein the PDE-I is sildenafil.
4. A method of any one of embodiments 1-3 wherein the time period is within
24 hours of exposure to a blast; within 1 hour of exposure to a blast; within 10 minutes of exposure to a blast; or within 5 minutes of exposure to a blast.
5. A method of any one of embodiments 1-4 wherein the blast creates a pressure wave of 10 psi or greater; 20 psi or greater; or 22 psi or greater.
6. A method of any one of embodiments 1-5 wherein the tinnitus is high-frequency tinnitus and/or high frequency hearing loss.
7. A method of any one of embodiments 1-6 wherein the therapeutic treatment reduces blast-induced tinnitus in the subject 0-6 weeks following exposure to the blast.
8. A method of any one of embodiments 1-7 wherein the therapeutic treatment reduces blast-induced tinnitus in the subject 0 weeks following exposure to the blast.
9. A method of any one of embodiments 1-8 wherein the therapeutic treatment reduces blast-induced tinnitus in the subject 1 week following exposure to the blast.
10. A method of any one of embodiments 1-9 wherein the therapeutic treatment reduces blast-induced tinnitus in the subject 2 weeks following exposure to the blast.
11. A method of any one of embodiments 1-10 wherein the therapeutic treatment reduces blast-induced tinnitus in the subject 3 weeks following exposure to the blast.
12. A method of any one of embodiments 1-11 wherein the therapeutic treatment reduces blast-induced tinnitus in the subject 4 weeks following exposure to the blast.
13. A method of any one of embodiments 1-12 wherein the therapeutic treatment reduces blast-induced tinnitus in the subject 5 weeks following exposure to the blast.
14. A method of any one of embodiments 1-13 wherein the therapeutic treatment reduces blast-induced tinnitus in the subject 6 weeks following exposure to the blast.
15. A method of any one of embodiments 1-14 wherein the therapeutic treatment reduces blast-induced tinnitus in the subject more than 6 weeks following exposure to the blast.
16. A method of any one of embodiments 1-8 wherein the therapeutic treatment reduces 26-28 kHz blast-induced tinnitus in the subject.
17. A method of any one of embodiments 1-16 wherein the therapeutic treatment reduces 6-8 kHz; 10-12 kHz; 14-16 kHz; 18-20 kHz; 22-24 kHz; and/or 26-28 kHz blast-induced tinnitus in the subject.
18. A method of any one of embodiments 1-17 wherein the therapeutic treatment reduces 6-8 kHz blast-induced tinnitus in the subject.
19. A method of any one of embodiments 1-18 wherein the therapeutic treatment reduces 10-12 kHz blast-induced tinnitus in the subject.
20. A method of any one of embodiments 1-19 wherein the therapeutic treatment reduces 14-16 kHz blast-induced tinnitus in the subject.
21. A method of any one of embodiments 1-20 wherein the therapeutic treatment reduces 18-20 kHz blast-induced tinnitus in the subject.
22. A method of any one of embodiments 1-21 wherein the therapeutic treatment reduces 22-24 kHz blast-induced tinnitus in the subject.
23. A method of any one of embodiments 1-22 wherein the therapeutic treatment reduces 26-28 kHz blast-induced tinnitus in the subject.
24. A method of any one of embodiments 1-23 wherein the therapeutic treatment reduces blast-induced hearing loss in the subject 0-6 weeks following exposure to the blast.
25. A method of any one of embodiments 1-24 wherein the therapeutic treatment reduces blast-induced hearing loss in the subject 0 weeks following exposure to the blast.
26. A method of any one of embodiments 1-25 wherein the therapeutic treatment reduces blast-induced hearing loss in the subject 1 week following exposure to the blast.
27. A method of any one of embodiments 1-26 wherein the therapeutic treatment reduces blast-induced hearing loss in the subject 2 weeks following exposure to the blast.
28. A method of any one of embodiments 1-27 wherein the therapeutic treatment reduces blast-induced hearing loss in the subject 3 weeks following exposure to the blast.
29. A method of any one of embodiments 1-28 wherein the therapeutic treatment reduces blast-induced hearing loss in the subject 4 weeks following exposure to the blast.
30. A method of any one of embodiments 1-29 wherein the therapeutic treatment reduces blast-induced hearing loss in the subject 5 weeks following exposure to the blast.
31. A method of any one of embodiments 1-30 wherein the therapeutic treatment reduces blast-induced hearing loss in the subject 6 weeks following exposure to the blast.
32. A method of any one of embodiments 1-31 wherein the therapeutic treatment reduces blast-induced hearing loss in the subject more than 6 weeks following exposure to the blast.
33. A method of any one of embodiments 1-32 wherein the therapeutic treatment reduces 6-8 kHz; 10-12 kHz; 14-16 kHz; 18-20 kHz; 22-24 kHz; and/or 26-28 kHz blast-induced hearing loss in the subject.
34. A method of any one of embodiments 1-33 wherein the therapeutic treatment reduces 6-8 kHz blast-induced hearing loss in the subject.
35. A method of any one of embodiments 1-34 wherein the therapeutic treatment reduces 10-12 kHz blast-induced hearing loss in the subject.
36. A method of any one of embodiments 1-35 wherein the therapeutic treatment reduces 14-16 kHz blast-induced hearing loss in the subject.
37. A method of any one of embodiments 1-36 wherein the therapeutic treatment reduces 18-20 kHz blast-induced hearing loss in the subject.
38. A method of any one of embodiments 1-37 wherein the therapeutic treatment reduces 22-24 kHz blast-induced hearing loss in the subject.
39. A method of any one of embodiments 1-38 wherein the therapeutic treatment reduces 26-28 kHz blast-induced hearing loss in the subject.

### Exemplary Embodiments

### Set 3

1. A method of treating high-frequency blast-induced tinnitus in a subject in need thereof comprising administering to the subject a therapeutically effective amount of a phosphodiesterase inhibitor (PDE-I) within a time period associated with exposure to a blast thereby providing a therapeutic treatment by reducing in the subject 26-28 kHz blast-induced tinnitus 3-6 weeks following exposure to the blast.
2. A method of embodiment 1 where the PDE-I is a PDE-5 inhibitor.
3. A method of embodiment 1 or 2 wherein the PDE-I is sildenafil.
4. A method of any one of embodiments 1-3 wherein the time period is within 24 hours of exposure to a blast; within 1 hour of exposure to a blast; within 10 minutes of exposure to a blast; or within 5 minutes of exposure to a blast.
5. A method of any one of embodiments 1-4 wherein the blast creates a pressure wave of 10 psi or greater; 20 psi or greater; or 22 psi or greater.
6. A method of any one of embodiments 1-5 wherein the therapeutic treatment reduces 26-28 kHz blast-induced tinnitus in the subject 3 weeks following exposure to the blast.
7. A method of any one of embodiments 1-6 wherein the therapeutic treatment reduces 26-28 kHz blast-induced tinnitus in the subject 4 weeks following exposure to the blast.
8. A method of any one of embodiments 1-7 wherein the therapeutic treatment reduces 26-28 kHz blast-induced tinnitus in the subject 5 weeks following exposure to the blast.
9. A method of any one of embodiments 1-8 wherein the therapeutic treatment reduces 26-28 kHz blast-induced tinnitus in the subject 6 weeks following exposure to the blast.
10. A method of treating high-frequency blast-induced tinnitus in a subject in need thereof comprising administering to the subject a therapeutically effective amount of a phosphodiesterase inhibitor (PDE-I) within a time period associated with exposure to a blast thereby providing a therapeutic treatment by reducing in the subject 18-20 kHz and/or 26-28 kHz blast-induced tinnitus 3-4 weeks following exposure to the blast.
11. A method of embodiment 10 where the PDE-I is a PDE-5 inhibitor.
12. A method of embodiment 10 or 11 wherein the PDE-I is sildenafil.
13. A method of any one of embodiments 10-12 wherein the time period is within 24 hours of exposure to a blast; within 10 hour of exposure to a blast; within 10 minutes of exposure to a blast; or within 5 minutes of exposure to a blast.
14. A method of any one of embodiments 10-13 wherein the blast creates a pressure wave of 10 psi or greater; 20 psi or greater; or 22 psi or greater.
15. A method of any one of embodiments 10-14 wherein the therapeutic treatment reduces 18-20 kHz blast-induced tinnitus in the subject 3 weeks following exposure to the blast.
16. A method of any one of embodiments 10-15 wherein the therapeutic treatment reduces 18-20 kHz blast-induced tinnitus in the subject 4 weeks following exposure to the blast.
17. A method of any one of embodiments 10-16 wherein the therapeutic treatment reduces 26-28 kHz blast-induced tinnitus in the subject 3 weeks following exposure to the blast.
18. A method of any one of embodiments 10-17 wherein the therapeutic treatment reduces 26-28 kHz blast-induced tinnitus in the subject 4 weeks following exposure to the blast.
19. A method of any one of embodiments 10-18 wherein the therapeutic treatment reduces 18-20 kHz and 26-28 kHz blast-induced tinnitus in the subject 3-4 weeks following exposure to the blast.
20. A method of treating hearing loss in a subject in need thereof comprising administering to the subject a therapeutically effective amount of a phosphodiesterase inhibitor (PDE-I) within a time period associated with exposure to a blast thereby providing a therapeutic treatment by reducing in the subject 6-8 kHz hearing loss 0-2 weeks following exposure to the blast; 14-16 kHz hearing loss 0-4 weeks following exposure to the blast and/or 18-20 kHz hearing loss 0-2 weeks and/or 4-6 weeks following exposure to the blast.
21. A method of embodiment 20 where the PDE-I is a PDE-5 inhibitor.
22. A method of embodiment 20 or 21 wherein the PDE-I is sildenafil.
23. A method of any one of embodiments 20-22 wherein the time period is within 24 hours of exposure to a blast; within 1 hour of exposure to a blast; within 10 minutes of exposure to a blast; or within 5 minutes of exposure to a blast.
24. A method of any one of embodiments 20-23 wherein the blast creates a pressure wave of 10 psi or greater; 20 psi or greater; or 22 psi or greater.
25. A method of any one of embodiments 20-24 wherein the therapeutic treatment reduces 6-8 kHz hearing loss in the subject 0 weeks following exposure to the blast.
26. A method of any one of embodiments 20-25 wherein the therapeutic treatment reduces 6-8 kHz hearing loss in the subject 1 week following exposure to the blast.
27. A method of any one of embodiments 20-26 wherein the therapeutic treatment reduces 6-8 kHz hearing loss in the subject 2 weeks following exposure to the blast.
28. A method of any one of embodiments 20-27 wherein the therapeutic treatment reduces 14-16 kHz hearing loss in the subject 0 weeks following exposure to the blast.
29. A method of any one of embodiments 20-28 wherein the therapeutic treatment reduces 14-16 kHz hearing loss in the subject 1 week following exposure to the blast.
30. A method of any one of embodiments 20-29 wherein the therapeutic treatment reduces 14-16 kHz hearing loss in the subject 2 weeks following exposure to the blast.
31. A method of any one of embodiments 20-30 wherein the therapeutic treatment reduces 14-16 kHz hearing loss in the subject 3 weeks following exposure to the blast.
32. A method of any one of embodiments 20-31 wherein the therapeutic treatment reduces 14-16 kHz hearing loss in the subject 4 weeks following exposure to the blast.
33. A method of any one of embodiments 20-32 wherein the therapeutic treatment reduces 18-20 kHz hearing loss in the subject 0 weeks following exposure to the blast.
34. A method of any one of embodiments 20-33 wherein the therapeutic treatment reduces 18-20 kHz hearing loss in the subject 1 week following exposure to the blast.
35. A method of any one of embodiments 20-34 wherein the therapeutic treatment reduces 18-20 kHz hearing loss in the subject 2 weeks following exposure to the blast.
36. A method of any one of embodiments 20-35 wherein the therapeutic treatment reduces 18-20 kHz hearing loss in the subject 4 weeks following exposure to the blast.
37. A method of any one of embodiments 20-36 wherein the therapeutic treatment reduces 18-20 kHz hearing loss in the subject 5 weeks following exposure to the blast.
38. A method of any one of embodiments 20-37 wherein the therapeutic treatment reduces 18-20 kHz hearing loss in the subject 6 weeks following exposure to the blast.
39. A method of treating high-frequency blast-induced tinnitus and hearing loss in a subject in need thereof comprising administering to the subject a therapeutically effective amount of a phosphodiesterase inhibitor (PDE-I) within a time period associated with exposure to a blast thereby providing a therapeutic treatment by reducing in the subject (i) 26-28 kHz blast-induced tinnitus 3-6 weeks following exposure to the blast (ii) 18-20 kHz and/or 26-28 kHz blast-induced tinnitus 3-4 weeks following exposure to the blast; and (iii) 6-8 kHz hearing loss 0-2 weeks following exposure to the blast; 14-16 kHz hearing loss 0-4 weeks following exposure to the blast and/or 18-20 kHz hearing loss 0-2 weeks and/or 4-6 weeks following exposure to the blast.
40. A method of embodiment 39 where the PDE-I is a PDE-5 inhibitor.
41. A method of embodiment 39 or 40 wherein the PDE-I is sildenafil.
42. A method of any one of embodiments 39-41 wherein the time period is within 24 hours of exposure to a blast; within 1 hour of exposure to a blast; within 10 minutes of exposure to a blast; or within 5 minutes of exposure to a blast.
43. A method of any one of embodiments 39-42 wherein the blast creates a pressure wave of 10 psi or greater; 20 psi or greater; or 22 psi or greater.
44. A method of any one of embodiments 39-43 wherein the therapeutic treatment reduces 26-28 kHz blast-induced tinnitus in the subject 3 weeks following exposure to the blast.
45. A method of any one of embodiments 39-44 wherein the therapeutic treatment reduces 26-28 kHz blast-induced tinnitus in the subject 4 weeks following exposure to the blast.
46. A method of any one of embodiments 39-45 wherein the therapeutic treatment reduces 26-28 kHz blast-induced tinnitus in the subject 5 weeks following exposure to the blast.
47. A method of any one of embodiments 39-46 wherein the therapeutic treatment reduces 26-28 kHz blast-induced tinnitus in the subject 6 weeks following exposure to the blast.
48. A method of any one of embodiments 39-47 wherein the therapeutic treatment reduces 18-20 kHz blast-induced tinnitus in the subject 3 weeks following exposure to the blast.
49. A method of any one of embodiments 39-48 wherein the therapeutic treatment reduces 18-20 kHz blast-induced tinnitus in the subject 4 weeks following exposure to the blast.
50. A method of any one of embodiments 39-49 wherein the therapeutic treatment reduces 26-28 kHz blast-induced tinnitus in the subject 3 weeks following exposure to the blast.
51. A method of any one of embodiments 39-50 wherein the therapeutic treatment reduces 26-28 kHz blast-induced tinnitus in the subject 4 weeks following exposure to the blast.
52. A method of any one of embodiments 39-51 wherein the therapeutic treatment reduces 18-20 kHz and 26-28 kHz blast-induced tinnitus in the subject 3-4 weeks following exposure to the blast.
53. A method of any one of embodiments 39-52 wherein the therapeutic treatment reduces 6-8 kHz hearing loss in the subject 0 weeks following exposure to the blast.
54. A method of any one of embodiments 39-53 wherein the therapeutic treatment reduces 6-8 kHz hearing loss in the subject 1 week following exposure to the blast.
55. A method of any one of embodiments 39-54 wherein the therapeutic treatment reduces 6-8 kHz hearing loss in the subject 2 weeks following exposure to the blast.
56. A method of any one of embodiments 39-55 wherein the therapeutic treatment reduces 14-16 kHz hearing loss in the subject 0 weeks following exposure to the blast.
57. A method of any one of embodiments 39-56 wherein the therapeutic treatment reduces 14-16 kHz hearing loss in the subject 1 week following exposure to the blast.
58. A method of any one of embodiments 39-57 wherein the therapeutic treatment reduces 14-16 kHz hearing loss in the subject 2 weeks following exposure to the blast.
59. A method of any one of embodiments 39-58 wherein the therapeutic treatment reduces 14-16 kHz hearing loss in the subject 3 weeks following exposure to the blast.
60. A method of any one of embodiments 39-59 wherein the therapeutic treatment reduces 14-16 kHz hearing loss in the subject 4 weeks following exposure to the blast.
61. A method of any one of embodiments 39-60 wherein the therapeutic treatment reduces 18-20 kHz hearing loss in the subject 0 weeks following exposure to the blast.
62. A method of any one of embodiments 39-61 wherein the therapeutic treatment reduces 18-20 kHz hearing loss in the subject 1 week following exposure to the blast.
63. A method of any one of embodiments 39-62 wherein the therapeutic treatment reduces 18-20 kHz hearing loss in the subject 2 weeks following exposure to the blast.
64. A method of any one of embodiments 39-63 wherein the therapeutic treatment reduces 18-20 kHz hearing loss in the subject 4 weeks following exposure to the blast.
65. A method of any one of embodiments 39-64 wherein the therapeutic treatment reduces 18-20 kHz hearing loss in the subject 5 weeks following exposure to the blast.
66. A method of any one of embodiments 39-65 wherein the therapeutic treatment reduces 18-20 kHz hearing loss in the subject 6 weeks following exposure to the blast.

The Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### Examples

### I. Methods

A. Animal Subjects.

Thirty adult Sprague Dawley rats (110 days old, 250-300 g) were purchased from Charles River Laboratories (Wilmington, Mass.). Three animals were initially excluded from the study due to poor startle reflex and two animals died from unrelated causes. Another animal was retrospectively removed from the study due to epistaxis immediately following the first blast and absence of startle responsiveness during post-blast testing. Of the remaining 24 animals, 10 were blast-exposed and treated with sildenafil (Treated group), 6 were blast-exposed but were given vehicle tap water (Untreated group), and 8 received sildenafil treatment but no blast exposure (Sham group). A sham-blasted group that did not receive sildenafil treatment was not included as the Sham group displayed relatively stable behavioral performance and hearing thresholds over time. All procedures were approved by the Institutional Animal Care and Use Committee at Wayne State University and were in accordance with the regulations of the Federal Animal Welfare Act. All efforts were made to minimize animal suffering, to reduce the number of animals used, and to utilize alternatives to in vivo techniques, if available.

B. Gap-Detection and Prepulse Inhibition Testing (Before Blast Exposure).

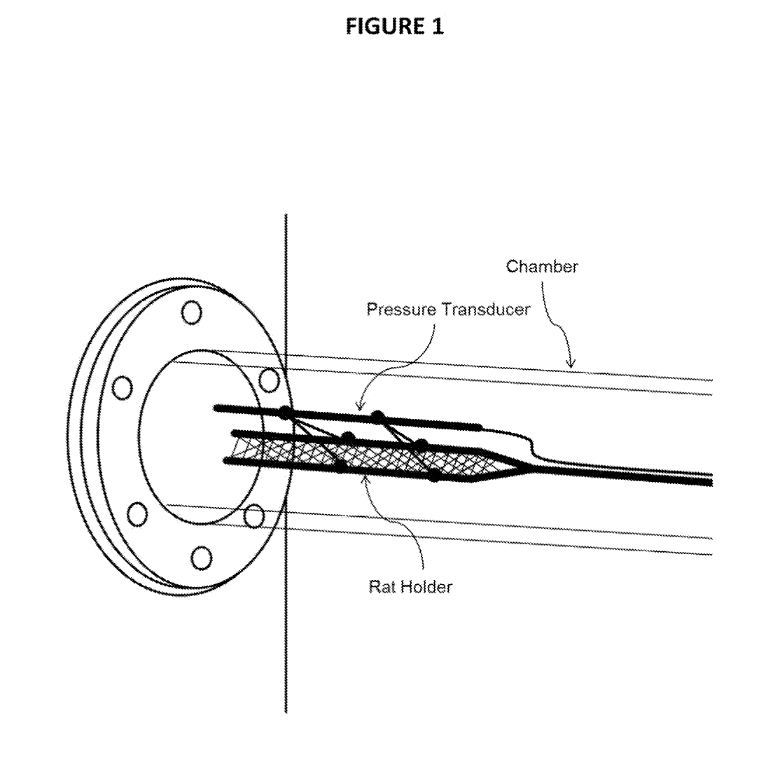

Animals underwent 8 rounds of behavior testing to stabilize baseline Gap-detection and PPI performance prior to blast or sham-blast exposure. Gap-detection and PPI tests were conducted using acoustic startle reflex hardware and software (Kinder Scientific, Poway, Calif.), as described elsewhere (Zhang, S. J. et al., Chinese J of Otorhinolaryngology Head and Neck Surg 46(10): 844-847, 2011; Luo H, et al., Neurosci Lett 26; 522, 2012; Pace, E. and Zhang, J. S. PLoS ONE 8:e75011, 2013). Briefly, each rat was placed in a custom-built polycarbonate restrainer (FIG. 1) and set inside a lit startle monitor cabinet equipped with two ceiling speakers for background sound/prepulses and startle stimuli. Restrainers were mounted on top of a platform connected to a piezoelectric transducer, which measured downward startle force. Acoustic stimuli and startle force were calibrated using a Newton impulse calibrator (Kinder Scientific) and a microphone (Model 4016; ACO Pacific, Belmont, Calif.).

During the Gap-detection procedure, rats were exposed to constant 60 dB sound pressure level (SPL) background noise consisting of 2 kHz bandpass signals from 2-4 kHz, 6-8 kHz, 8-10 kHz, 10-12 kHz, 14-16 kHz, 18-20 kHz, or 26-28 kHz, or BBN (2-30 kHz). They were subjected to either a 50 ms white noise burst startle stimulus (startle only; dominating plateau from 3-37 kHz with energy up to 97.5 kHz) presented at 115 dB or the startle stimulus preceded by a 40 ms silent period beginning 90 ms before the startle stimulus (Gap). For each testing session, rats were presented 8 times with the startle only and Gap conditions for each frequency bandpass signal and BBN. The PPI procedure was identical to the Gap-detection procedure except that no background sound was presented. Rats were subjected to either the startle stimulus alone (startle only) or the startle stimulus preceded by a 40 ms prepulse beginning 90 ms before the startle stimulus. Prepulses (60 dB, SPL) consisted of the same frequencies as those used for background noise.

A two-minute acclimatization period was given at the beginning of each session, followed by two startle stimuli presented without background sound to trigger any initial, exaggerated startle reflexes. Gap-detection and PPI testing sessions lasted a little over thirty minutes each.

C. Auditory Brainstem Response (ABR) Recording (Before Blast Exposure).

ABRs were recorded to evaluate hearing thresholds. Each rat was anesthetized using a mixture of air (0.41/min) and isoflurane (2-3%, v/v) and placed in a prone position with its head fixed to a stereotaxic frame. Body temperature was maintained using a warming blanket connected to a thermostatic controller (Harvard Instruments, Holliston, Mass.). Acoustic stimuli consisted of 0.1 ms clicks or 10 ms pure-tone bursts presented at 4, 8, 12, 16, 20, or 28 kHz and delivered through a speaker tube inserted into the external auditory canal. Three subcutaneous platinum-coated tungsten electrodes were used to record ABR waveforms, with the reference electrode inserted below the pinna ipsilateral to the speaker tube, the grounding electrode inserted below the contralateral pinna, and the recording electrode inserted at the vertex. Evoked potentials were bandpass-filtered at 300-3000 Hz, notch-filtered at 60 Hz, and averaged 300-400 times for clicks and tone-bursts, respectively. Data were recorded using BioSigRP® and SigGenRP® software (TDT, Alachua, Fla.) installed on an IBM computer connected to System 3 TDT workstation.

D. Blast Exposure.

To induce tinnitus and auditory impairment, rats underwent three consecutive blast exposures. The blast shock waves were generated by a custom-built shock tube (ORA Inc. Fredericksburg, Va.) located in the Biomedical Engineering Building at Wayne State University. Peak static overpressure of 22 psi was produced with compressed helium and calibrated Mylar sheets (GE Richards Graphics Supplies Inc., Landsville, Pa.) to produce a free field blast wave. Prior to each exposure, rats were anesthetized with a mixture of isoflurane (3%) and 0.6 L/min of oxygen for 6 minutes. While anesthetized, the rat was harnessed to a sled and positioned 109 cm inside the open end of the shock tube in a rostro-cephalic orientation towards the oncoming shock waves. The right ear was occluded with an earplug (Mack's®, McKeon Products, Warren, Mich.) for protection against noise trauma, so that responsivity to acoustic stimuli during Gap-detection and PPI testing could be retained.

Immediately after each blast exposure, the still anesthetized rat was placed on its back and monitored for latency to surface right, an indirect marker of unconsciousness. Surface righting was defined as settling on all four paws. An average of five minutes was given between successful surface righting and anesthesia induction for the subsequent blast exposure, during which time rats were transferred to polycarbonate cages. Immediately after successful surface righting following the last blast exposure, sildenafil was administered to Treated group and the Sham group, while a tap water vehicle was given to the Untreated group.

E. Sildenafil Administration.

Sildenafil tablets (100 mg) were crushed, dissolved in tap water and administered once a day at a 10 mg/kg dosage via oral gavage for 7 days after blast exposure. A longer and continuous treatment regimen was not selected because PDE-5 inhibitors have reportedly contributed to hearing loss. The Untreated group received a similar volume of tap water. Curved, stainless steel, ball-nosed feeding needles (20 ga×3″, Popper and Sons, New Hyde Park, N.Y.) were used to deliver the drug orally and were cleaned with tap water after use. Rats were exposed to the feeding needle several times prior to drug administration to habituate them to the oral gavage procedure and reduce stress. After the initial 7-day round of treatment, rats underwent one week without treatment to assess washout effect. Because the data indicated a mild therapeutic effect, the same Treated group was then given a second 7-day round during the third week post-blast. The pharmacokinetic profile of sildenafil shows detectability in blood plasma within 5 minutes upon oral intake and Tₘₐₓ values within 11 minutes.

### II. Results & Analysis I

A. Surface Righting Latency.

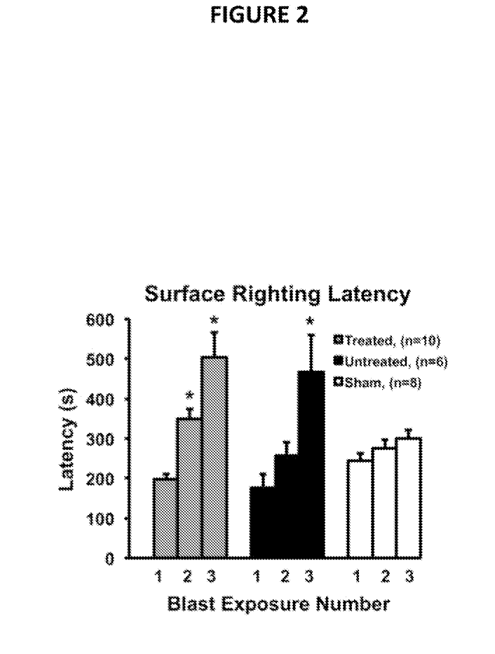

Following blast exposures, rats were measured for surface righting latency as an index of unconsciousness. Although there were no statistically significant differences between the Treated, Untreated and Sham groups after any of the three blasts, the Treated group and the Untreated group displayed a significantly longer latency for surface righting after their third blast compared to their first (Treated, p<0.001; Untreated, p=0.029; paired t-test), indicating a dose-dependent effect of blast on unconsciousness (FIG. 2). The Sham group showed no difference between their first and third post-blast surface righting latencies (p=0.103; paired t-test).

B. Gap-Detection, PPI, and ABR Testing (after Blast Exposure).

Gap-detection and PPI testing were performed one hour following the last blast exposure and for 8 weeks afterward to track the progression of tinnitus and hearing loss. ABRs were performed for each rat on the day of blast exposure and at 1, 3, and 6 weeks post-blast to monitor recovery of hearing thresholds.

C. Data Analysis of Gap and PPI Ratio Results.

Behavioral data prior to blast exposure was pooled for all 24 rats to establish a “pre-blast” baseline in order to account for natural individual differences in behavioral performance among the test groups and to achieve a more uniform baseline reflective of a much larger population. Behavioral data was then compared for the Treated group, the Untreated group, and the Sham group versus pooled pre-blast data.

D. Effect of Blast Exposure.

To study the effects of the blast exposure on tinnitus, Untreated group Gap data was compared to pre-blast Gap data at 0-2 weeks, 2-4 weeks, and 4-6 weeks post-blast intervals. To study the effects of blast exposure on hearing loss, PPI data was compared between the Untreated group and the pre-blast data at 0-2 weeks, 2-4 weeks, and 6-8 weeks. Furthermore, ABR thresholds were compared between the pre-blast and the post-blast Untreated group.

E. Gap-Detection Ratio Results.

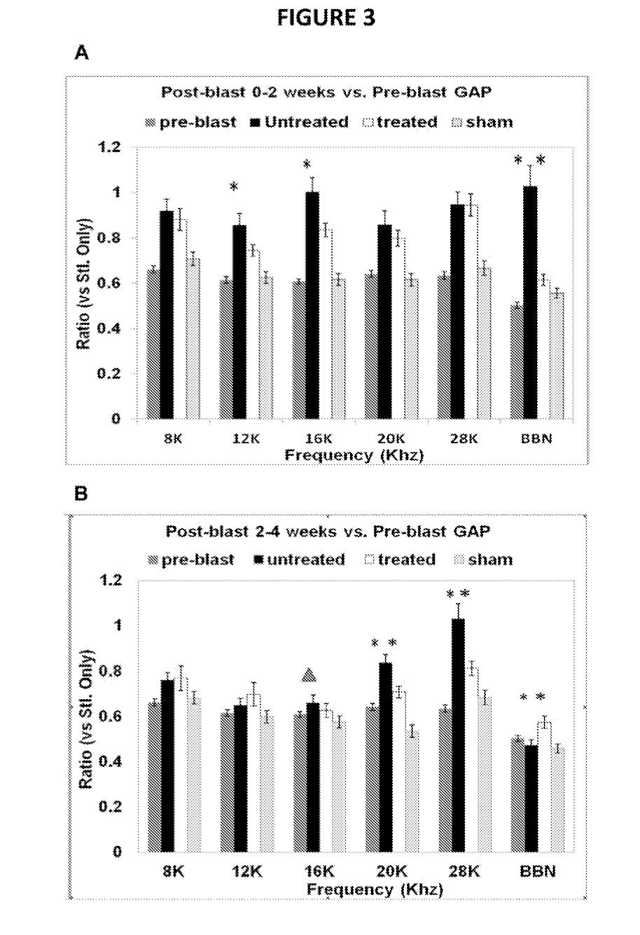

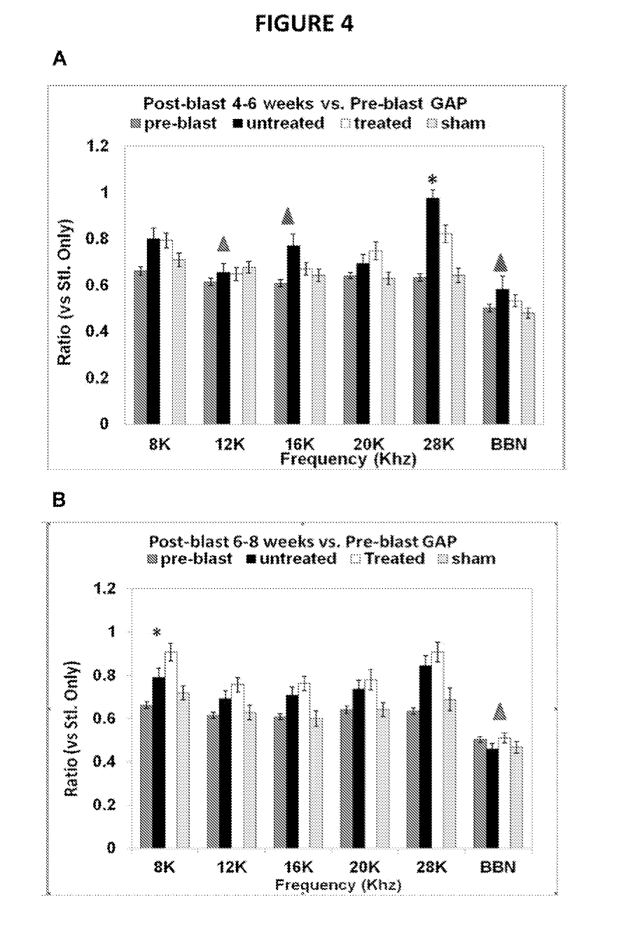

For the results described in FIGS. 3 and 4, a Gap-startle to startle-only ratio greater than or equal to 0.8 indicates tinnitus positive symptoms. In the first two weeks after blast exposure, both the Untreated and the Treated groups show significant deficits in Gap-detection. However, the Untreated group shows significantly higher Gap deficits compared to the Treated group at 10-12 kHz (p<0.05), 14-16 kHz (p<0.05), and BBN (p<0.01). In fact, the Untreated group showed worse group average Gap behavior compared to the Treated group at all tested frequencies in the first 2 weeks post-blast (FIG. 3A).

At 2-4 weeks post-blast (FIG. 3B), the Untreated group continued to show significant Gap-detection deficits at 18-20 kHz (p<0.01) and 26-28 kHz (p<0.01) relative to the Treated group. At 4-6 weeks, the Gap ratios at most frequencies appeared to be normalized, but 26-28 kHz still showed markedly elevated Gap ratios for the Untreated group (p<0.05). The data indicates that the therapeutic effect of sildenafil targets high frequency tinnitus symptoms for a significant period of time following blast injury.

At 6-8 weeks (FIG. 4B), there is no significant difference between the Treated and Untreated groups, except at 8 kHz where the Treated group has a deteriorated Gap-detection ratio compared to the Untreated group (p<0.05). Additionally, at BBN the Gap ratios for both the Treated and the Untreated groups were reduced back to pre-blast levels.

These results suggest that sildenafil conveyed a therapeutic effect on the various stages of blast-induced tinnitus. This therapeutic effect is maintained up to week 6, whereas the Untreated group exhibited marked deterioration in Gap-detection at both low-end and high-end frequencies. The Sham group which received the drug, but no blast, exhibited relatively stable Gap-detection throughout the study, indicating the drug itself did not produce tinnitus symptoms (FIGS. 3A, 3B, 4A and 4B).

F. PPI Ratio Results.

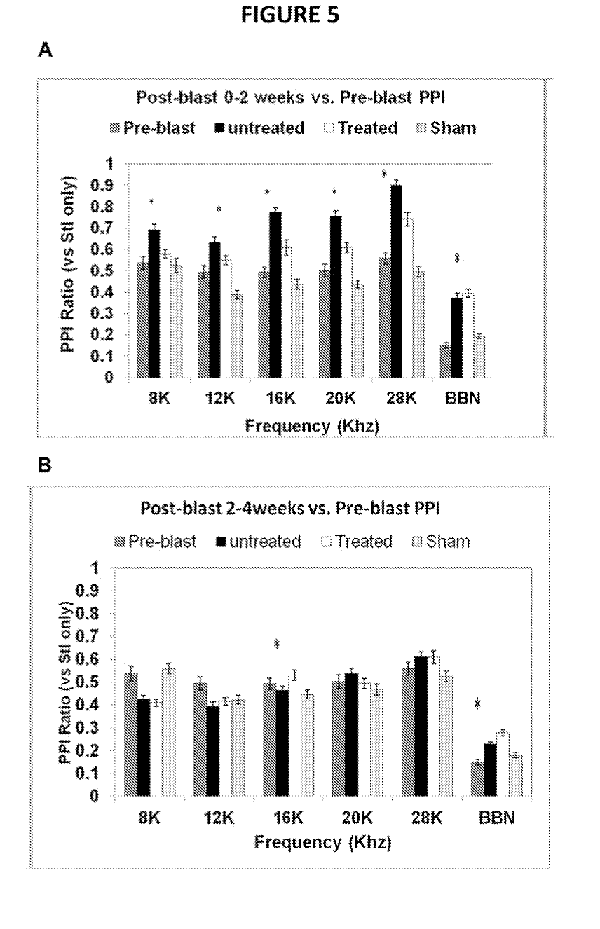

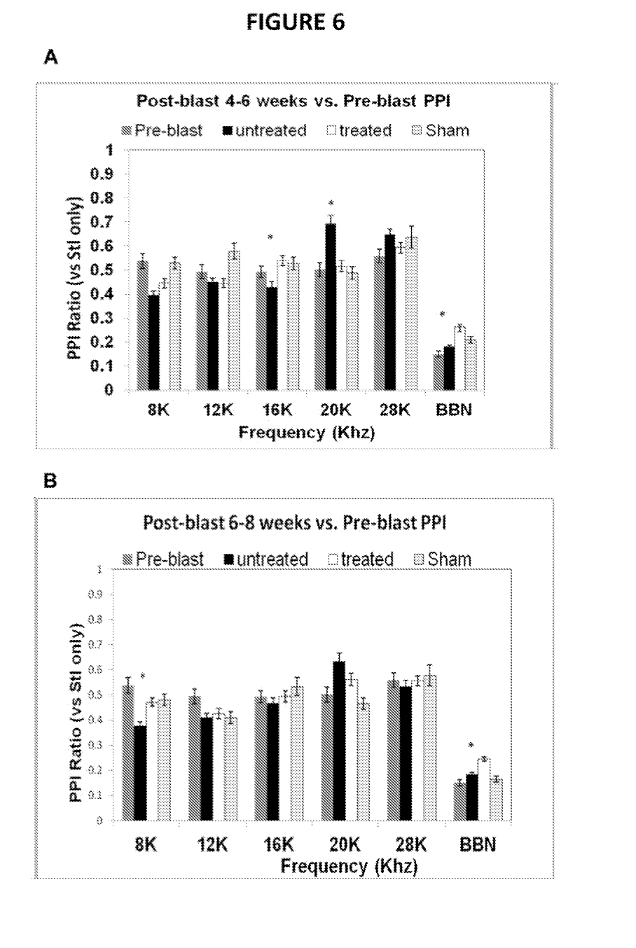

PPI results allow assessment of hearing loss when used in combination with ABR thresholds. Analysis of PPI ratios in FIGS. 5A, 5B, 6A, and 6B demonstrates that behavioral performance on the PPI testing paradigm shows elevated ratios at all tested frequencies relative to the pre-blast baseline, indicating hearing loss was present in the first 2 weeks after blast. Furthermore, 6-8 kHz (p<0.05), 14-16 kHz (p<0.05), and 18-20 kHz (p<0.01) each show significantly higher ratio elevations in the Untreated group compared to the Treated group. These results suggest that sildenafil affords protection against blast-induced hearing loss at the initial post-blast phase (FIG. 5A).

At 2-4 weeks, the PPI startle ratio for the Untreated group is reduced to pre-blast levels. There is no significant difference between the Treated and Untreated groups across most frequencies except 14-16 kHz and BBN (FIG. 5B). At 4-6 weeks, there is no significant difference between the Treated and Untreated PPI startle ratios except at 14-16 kHz, 18-20 kHz, and BBN frequencies (FIG. 6A). The Treated group has a lower PPI ratio compared to the Untreated group at 18-20 kHz, whereas the Untreated group has a lower PPI ratio a 14-16 kHz BBN frequency.

Finally, 6-8 week PPI results (FIG. 6B) indicate that behavior in response to prepulse stimulus has largely stabilized for most frequencies. The Treated group does, however, show significantly worse startle response at 8 kHz (p<0.05) and BBN (p<0.01).

G. Wave 1 Amplitude Data.

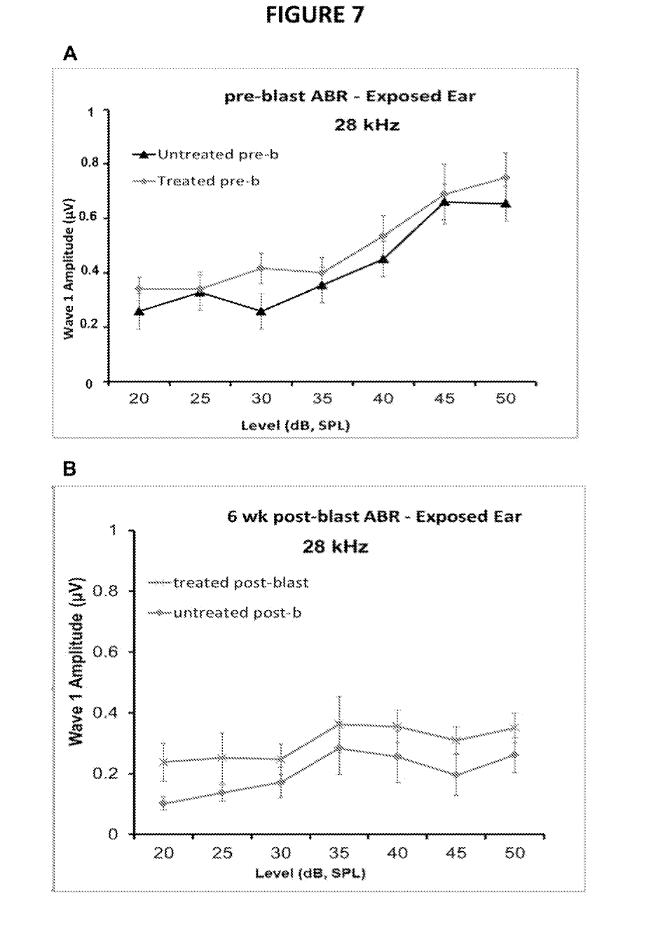

There is a significant reduction in wave 1 amplitudes in human subjects suffering from tinnitus despite a seemingly normal audiogram. In order to assess the possibility of underlying wave 1 amplitude reduction despite recovery of ABR thresholds, wave 1 amplitudes for 28 kHz frequency were measured at pre-blast and at 6 weeks post-blast and compared between the Treated and Untreated groups (FIGS. 7A and 7B). FIG. 7 shows significant reduction in wave 1 amplitudes at 6 weeks post-blast for 28 kHz, which happens to be the only frequency at which significant tinnitus persists at 6 weeks. Reduction in wave 1 amplitude is expected due to auditory nerve fiber damage in response to blast exposure.

According to the results described herein, the Untreated group exhibits a return to baseline levels of ABR thresholds, albeit at a later time point compared to the Treated group. Thus, without being bound by theory, sildenafil cytoprotection affords the ability of the organ of corti to repair itself or prevent exacerbation of injury following blast exposure, ultimately resulting in earlier recovery from hearing loss. Similar to the findings described herein, a temporary elevation of hearing thresholds has been observed in a recent study of rats in the first 24 hours following noise exposure (Kujawa S. G. and Liberman M. C., J Neurosci 29:14077-14085, 2009). The authors attributed this temporary shift in thresholds to swelling of cochlear nerve terminals that resolved by 2 weeks, which is also in line with the findings disclosed herein. It was suggested that recovery of response thresholds may mask underlying neuronal degeneration (deafferentation) following noise exposure. For example, permanent loss of 50-60% of auditory nerve fibers was observed despite nearly full recovery of ABR thresholds. This underlying “hidden” manifestation of hearing loss is ascribed to loss of cochlear neurons following noise-induced trauma and is measured as a decrease in wave 1 amplitudes. The drop in wave 1 amplitude is seen at higher frequencies, whereas lower frequency nerve fibers seem to have a more robust recovery after blast-induced injury. In contrast, the high frequency fibers do not recover over time and seem to be permanently modified. In the studies described herein, it was determined that 6 weeks after the triple blast exposure, the wave 1 amplitude decreased by 45% in both the Treated group and the Untreated groups at 28 kHz. These findings strongly suggest that the triple-blast model, described herein, caused nerve injury to cochlear nerve fibers despite the rapid recovery in ABR thresholds in both groups. This “deafferentation” injury at higher frequency persisted up to 6 weeks following blast exposure. The results confirm the efficacy of the triple blast in inducing permanent cochlear nerve damage, more accurately mimicking the experience of patients who suffer from tinnitus and hearing loss following TBI.

### III. Results & Analysis II

A. Data Analysis—Gap-Detection Ratio Change, PPI Ratio Change, Surface Righting Latency, and ABR Thresholds.

Gap-detection data were divided into ratios, as previously described (Zhang, S. J. et al., Chinese J of Otorhinolaryngology Head and Neck Surgery 46:844-847, 2011; Luo H, et al., Neurosci Lett 26; 522, 2012; Mao, J. C. et al., J Neurotrauma 29(2): 430-444, 2012; Pace, E. and Zhang, J. S. PLoS ONE 8:e75011, 2013). Briefly, for each frequency or BBN, the response to the Gap condition was divided by the mean response to the associated startle only condition, resulting in a ratio value between 0 and 1. A value close to 0 would indicate strong suppression of the startle reflex in response to silent gaps, and thus healthy status, whereas a value close to 1 would signify little suppression in response to the gap, indicating tinnitus. To determine whether blast exposure had an effect on Gap-detection, the pre-blast Gap ratio was subtracted from the post-blast ratio and the percentage of change from pre-blast exposure was calculated. This was done for each of the 3 groups for several post-blast time points, including the first and second rounds of sildenafil treatment (1 and 3 weeks post-blast, respectively), and 4, 6 and 7 weeks post-blast. Two to three tests per rat were included in each time point, and the percentage of change was compared between the Treated, Untreated, and Sham groups. A higher percentage of change compared to the Sham group would indicate blast-induced Gap impairment. A lower percentage of change in the Treated group compared to Untreated group would indicate Gap improvement due to sildenafil treatment. PPI data were analyzed in the same manner, except that PPI ratios were calculated and used instead of Gap ratios, and upward or downward changes in PPI data would indicate hearing loss or improvement, respectively.

Recently, it has been shown that a reduction in overall startle force can raise Gap and PPI ratios, potentially leading to false tinnitus diagnoses. To account for this, the pre-blast startle force in response to the startle only condition was subtracted from post-blast startle force and the percentage of startle force change from pre-blast was computed. This was done for the Gap-detection startle only condition (with background noise) and the PPI startle only condition (without background noise) and the change in startle force was compared between the Treated, Untreated, and Sham groups.

Surface righting latency was measured after each of the three blasts for each rat and also compared between the three groups. Longer surface righting latencies indicated longer periods of unconsciousness.

Finally, ABR threshold shifts were compared between groups by subtracting the pre-blast threshold from the post-blast threshold. Threshold shifts were determined for each recording time point, including post-blast day 0, and post-blast week 1, 3 and 6. Thresholds were considered to be the lowest sound intensity at which a distinct portion of the biological ABR waveform remained visible.

All between-group comparisons on data from Gap/PPI and ABR testing were conducted using one-way ANOVA with post-hoc Bonferroni to adjust alpha values. A second level of Bonferroni adjustment was added to control for the 5 frequencies (in addition to BBN) tested at each time point. If overall significance was determined by Bonferroni-adjusted F-tests, group-group comparisons were considered significant when p<0.05. Statistics from Gap/PPI and ABR data were organized into tables (Tables 1-6).

Immediately following blast exposure, both the Treated and Untreated groups exhibited behavioral evidence of tinnitus and hearing loss as well as bilateral hearing threshold shifts at all frequencies. All blasted rats as a whole showed delayed surface to right latency, suggesting that blast exposure contributed to unconsciousness. The Treated group displayed 26-28 kHz tinnitus suppression from 3-6 weeks post-blast, after which high-frequency tinnitus reemerged. They also displayed less hearing impairment on some measurements compared to the Untreated group during the first week post-blast, although this disappeared by the third week. Interestingly, the Untreated group did not exhibit an overall decrease in startle force like the Treated group, but occasionally showed increased startle force, suggesting possible hyperacusis-like precepts. Taken together, the described results indicate that sildenafil suppressed tinnitus and reduced hearing impairment in a time- and injury-dependent fashion.

B. Gap-Detection and PPI—Ratio Change.

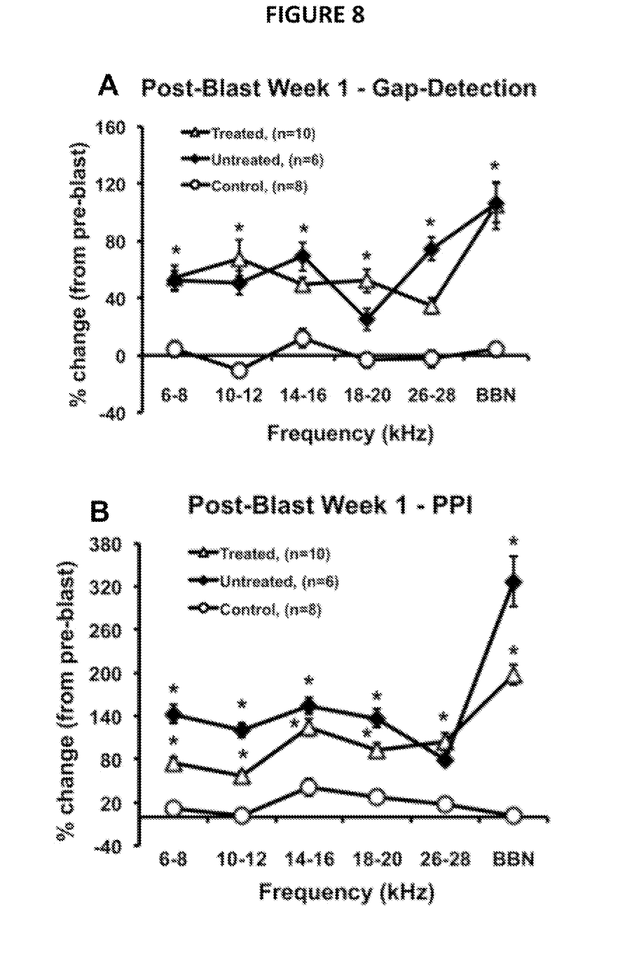

Gap-detection and PPI testing were conducted to assess the therapeutic effect of sildenafil on blast-induced tinnitus and hearing loss (FIGS. 8-12).

At 1 week post-blast, both the Treated and Untreated groups exhibited significant upward percent change in Gap and PPI ratios, indicative of impairment, at all frequencies compared to the Sham group. Therefore, sildenafil treatment did not prevent immediate blast-induced tinnitus or hearing loss. Compared to the Untreated group, Treated rats showed worse Gap impairment at 18-20 kHz while the Untreated rats showed significantly worse Gap impairment at 26-28 kHz (FIG. 8A; statistics in Table 1) and worse PPI ratios at 6-12 kHz, 18-20 kHz, and BBN (FIG. 8B; statistics in Table 2) compared to Treated rats. Greater Gap impairment at 18-20 kHz for the Treated group and 26-28 kHz for the Untreated group implies stronger tinnitus at those frequencies for those groups, respectively. Worse PPI performance at several frequencies in the Untreated group indicated greater overall hearing impairment post-blast, suggesting that while sildenafil treatment exerted no preventative effects, it still significantly reduced hearing loss.

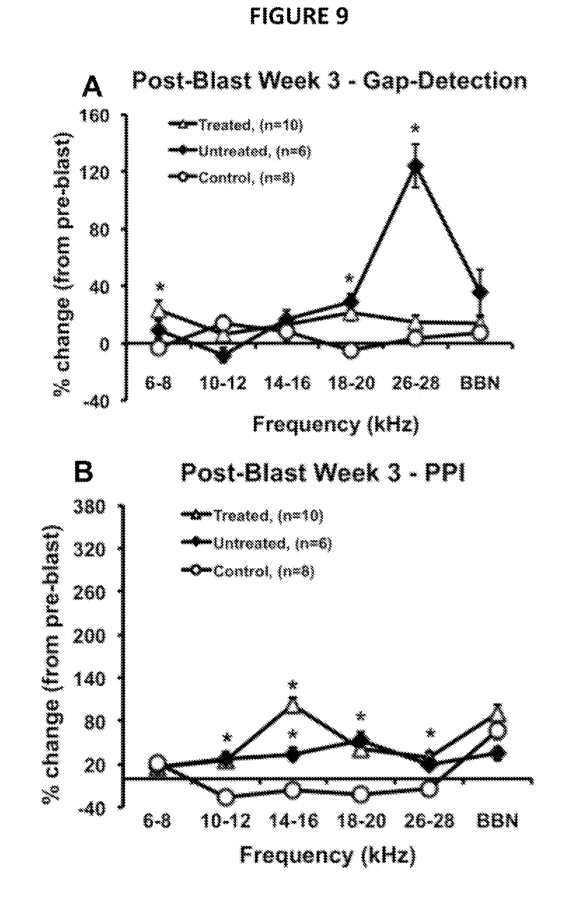

By 3 weeks post-blast, 18-20 kHz tinnitus and robust 26-28 kHz tinnitus persisted in Untreated rats (FIG. 9A), in addition to hearing loss from 10-28 kHz (FIG. 9B). The Treated group demonstrated tinnitus at 6-8 kHz and 18-20 kHz, but showed tinnitus suppression at 26-28 kHz, and hearing loss from 10-28 kHz. Interestingly, although the Treated group demonstrated the worst impairment at 14-16 kHz PPI, this was not associated with worse impairment at 14-16 kHz Gap. This may implicate complicated effects from TBI and/or a difference between Gap-detection and PPI neurocircuitry and consequent functioning.

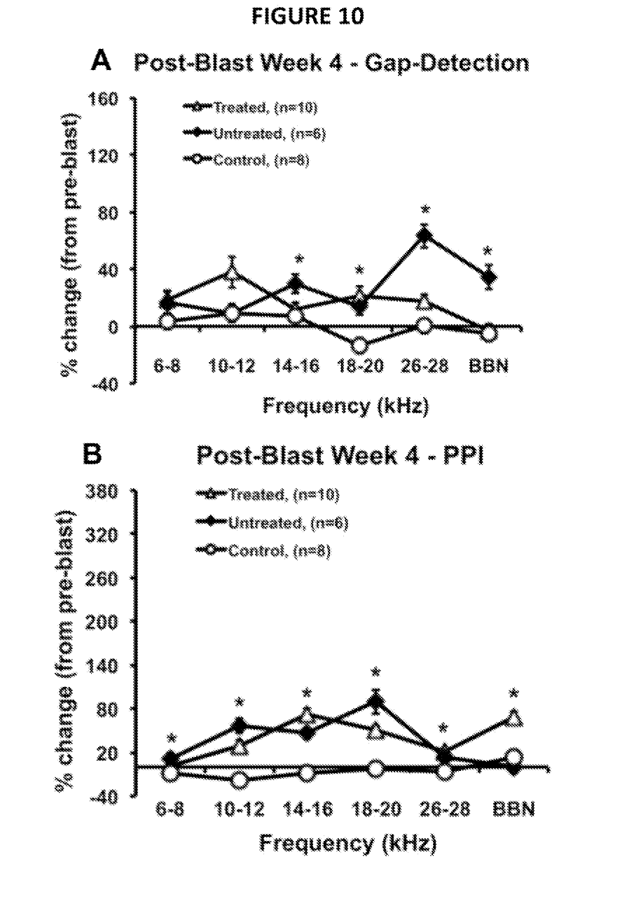

At 4 weeks post-blast, Untreated rats showed tinnitus from 14-28 kHz and BBN, while the Treated group exhibited tinnitus at 18-20 kHz but tinnitus suppression at all other frequencies (FIG. 10A). The Untreated group also demonstrated hearing loss from 18-20 kHz and BBN, while the Treated group showed deficits from 10-28 kHz (FIG. 10B).

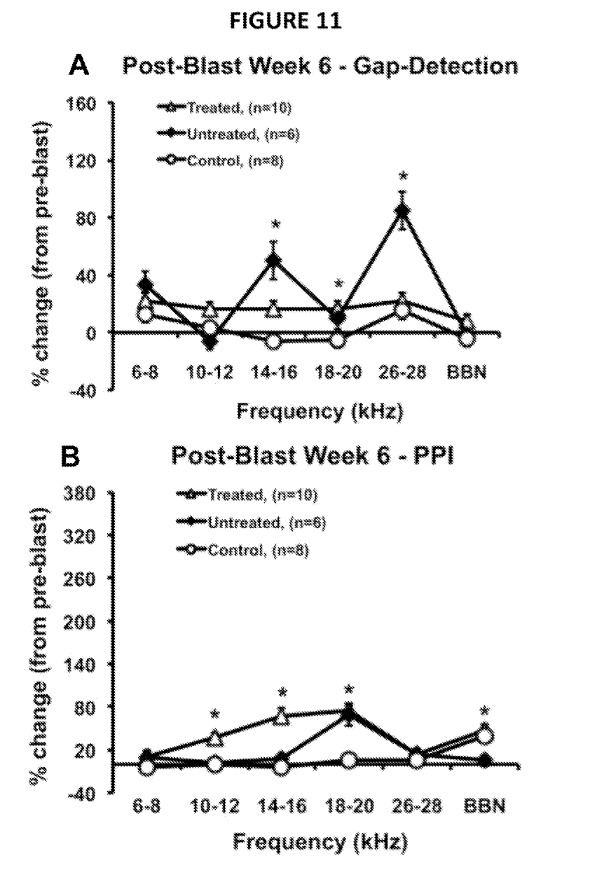

Six weeks following blast exposure, Untreated rats demonstrated tinnitus at 14-16 and 26-28 kHz (FIG. 11A) and hearing loss at 18-20 kHz (FIG. 11B). Treated rats, meanwhile, exhibited tinnitus from 14-20 kHz and hearing loss from 10-20 kHz. It should be noted that the Untreated group did not show impairment at 10-12 kHz or 14-16 kHz PPI, although these frequencies are impaired at all other time points. This may reflect increased sensitivity to these frequencies during this time point.

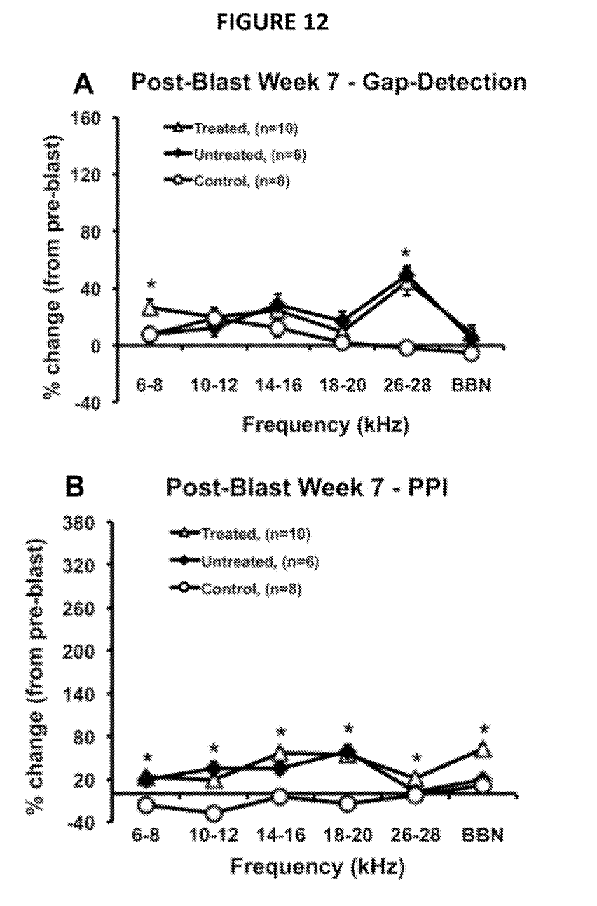

Lastly, at 7 weeks post-blast, Untreated rats maintained tinnitus at 26-28 kHz (FIG. 12A) and hearing loss from 6-20 kHz (FIG. 12B), while Treated rats exhibited tinnitus at 6-8 kHz and 26-28 kHz and hearing loss from 6-28 kHz and BBN.

TABLE 1
Statistical analysis - Gap data
GAP RATIO

| | 6-8 kHz | 10-12 kHz | 14-16 kHz | 18-20 kHz | 26-28 kHz | BBN |
|---|---|---|---|---|---|---|
| PB1Wk | F_{(2,490)} = | F_{(2,496)} = | F_{(2,497)} = | F_{(2,489)} = | F_{(2,485)} = | F_{(2,490)} = |
| | 10.275 | 12.442 | 14.854 | 12.133 | 26.448 | 12.481 |
| | T-U *T-S | T-U *T-S | T-U *T-S | *T-U *T-S | *T-U *T-S | T-U *T-S |
| | *U-S | *U-S | *U-S | U-S | *U-S | *U-S |
| PB3Wk | F_{(2,540)} = | F_{(2,538)} = | F_{(2,531)} = | F_{(2,531)} = | F_{(2,531)} = | F_{(2,531)} = |
| | 5.014 | 4.229 | 0.357 | 9.233 | 61.408 | 2.539 |
| | T-U *T-S | T-U T-S | T-U T-S | T-U *T-S | *T-U T-S | T-U T-S |
| | U-S | *U-S | U-S | *U-S | *U-S | U-S |
| PB4Wk | F_{(2,477)} = | F_{(2,474)} = | F_{(2,476)} = | F_{(2,466)} = | F_{(2,475)} = | F_{(2,477)} = |
| | 1.331 | 3.538 | 3.637 | 8.526 | 26.294 | 11.387 |
| | T-U T-S | T-U T-S | T-U T-S | T-U *T-S | *T-U T-S | *T-U T-S |
| | U-S | U-S | *U-S | *U-S | *U-S | *U-S |
| PB6Wk | F_{(2,472)} = | F_{(2,465)} = | F_{(2,462)} = | F_{(2,471)} = | F_{(2,462)} = | F_{(2,464)} = |
| | 2.202 | 4.070 | 12.449 | 3.994 | 19.737 | 1.423 |
| | T-U T-S | *T-U T-S | *T-U T-S | T-U *T-S | *T-U T-S | T-U T-S |
| | U-S | U-S | *U-S | U-S | *U-S | U-S |
| PB7Wk | F_{(2,514)} = | F_{(2,513)} = | F_{(2,502)} = | F_{(2,511)} = | F_{(2,508)} = | F_{(2,503)} = |
| | 4.907 | 0.453 | 1.908 | 1.459 | 14.040 | 1.549 |
| | *T-U *T-S | T-U T-S | T-U T-S | T-U T-S | T-U *T-S | T-U T-S |
| | U-S | U-S | U-S | U-S | *U-S | U-S |

TABLE 2
Statistical analysis - PPI data
PPI RATIO

| | 6-8 kHz | 10-12 kHz | 14-16 kHz | 18-20 kHz | 26-28 kHz | BBN |
|---|---|---|---|---|---|---|
| PB1Wk | F_{(2,494)} = | F_{(2,491)} = | F_{(2,493)} = | F_{(2,485)} = | F_{(2,484)} = | F_{(2,487)} = |
| | 39.167 | 44.518 | 17.074 | 21.880 | 15.240 | 49.933 |
| | *T-U *T-S | *T-U *T-S | T-U *T-S | T-U *T-S | *T-U *T-S | *T-U *T-S |
| | *U-S | *U-S | *U-S | *U-S | *U-S | *U-S |
| PB3Wk | F_{(2,410)} = | F_{(2,410)} = | F_{(2,410)} = | F_{(2,412)} = | F_{(2,409)} = | F_{(2,405)} = |
| | 0.135 | 23.110 | 52.802 | 28.934 | 12.696 | 4.9630 |
| | T-U T-S | T-U *T-S | *T-U *T-S | T-U *T-S | T-U *T-S | *T-U T-S |
| | U-S | *U-S | *U-S | *U-S | *U-S | U-S |
| PB4Wk | F_{(2,529)} = | F_{(2,535)} = | F_{(2,523)} = | F_{(2,532)} = | F_{(2,535)} = | F_{(2,528)} = |
| | 3.653 | 26.070 | 39.570 | 21.545 | 6.940 | 26.530 |
| | T-U *T-S | *T-U *T-S | *T-U *T-S | *T-U *T-S | T-U T-S | *T-U T-S |
| | U-S | *U-S | *U-S | *U-S | *U-S | *U-S |
| PB6Wk | F_{(2,463)} = | F_{(2,462)} = | F_{(2,462)} = | F_{(2,470)} = | F_{(2,462)} = | F_{(2,448)} = |
| | 1.913 | 8.278 | 19.838 | 15.216 | 0.694 | 6.096 |
| | T-U T-S | *T-U *T-S | *T-U *T-S | T-U *T-S * | T-U T-S | *T-U T-S |
| | U-S | U-S | U-S | U-S | U-S | *U-S |
| PB7Wk | F_{(2,549)} = | F_{(2,549)} = | F_{(2,553)} = | F_{(2,541)} = | F_{(2,556)} = | F_{(2,532)} = |
| | 14.427 | 31.395 | 22.684 | 38.323 | 5.775 | 21.369 |
| | T-U *T-S | T-U *T-S | T-U *T-S | T-U *T-S | *T-U T-S | *T-U *T-S |
| | *U-S | *U-S | *U-S | *U-S | U-S | U-S |

C. Gap-Detection and Prepulse Inhibition—Startle Force Change.

Changes in startle force following blast exposure were also monitored, in part to determine whether they could account for changes in ratio values.

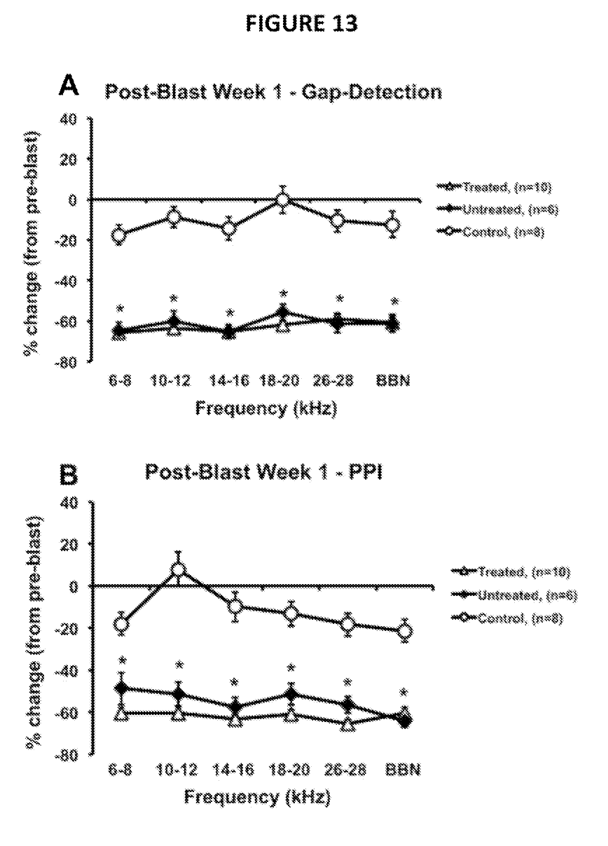

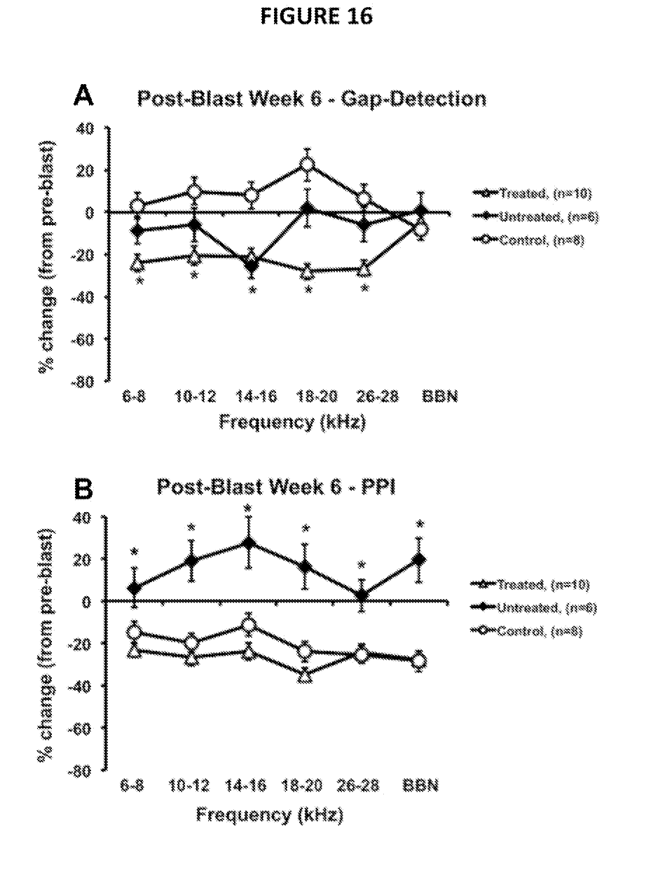

Following blast exposure, the Treated group showed significantly decreased startle force in response to the startle only condition with background noise (Gap-detection test) and without background noise (PPI test) across almost all time points compared to the Untreated and Sham groups (FIGS. 13-17). The exceptions were the 1 week post-blast time point, where Treated and Untreated rats sustained similar reductions in startle force during all background noises (FIG. 13A; statistics in Table 3), and without background noise (FIG. 13B; statistics in Table 4) compared to the Sham group. At 6 weeks post-blast, the Treated group showed similar startle force reduction without background noise compared to the Sham group, mostly due to a transient reduction in startle force for the Sham group (FIG. 16).

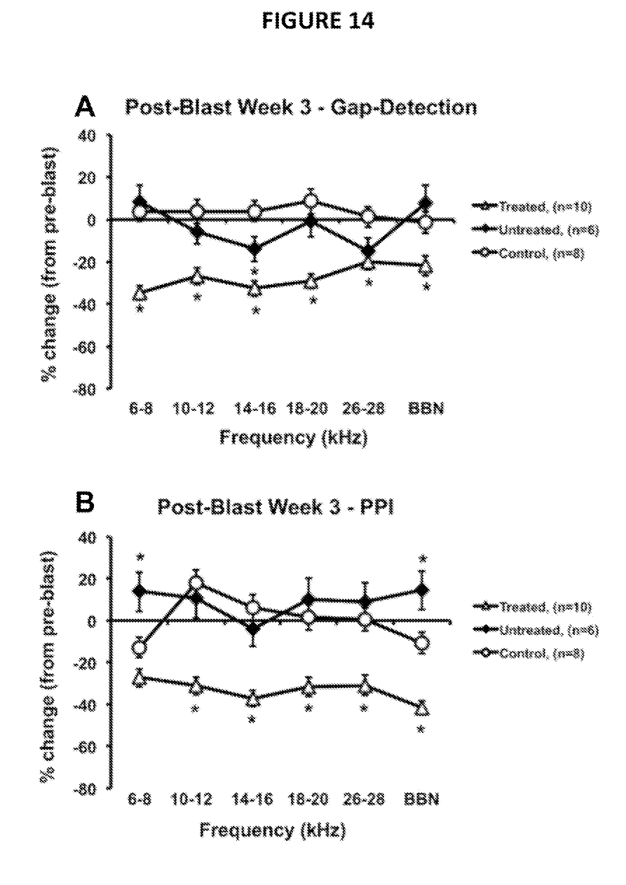

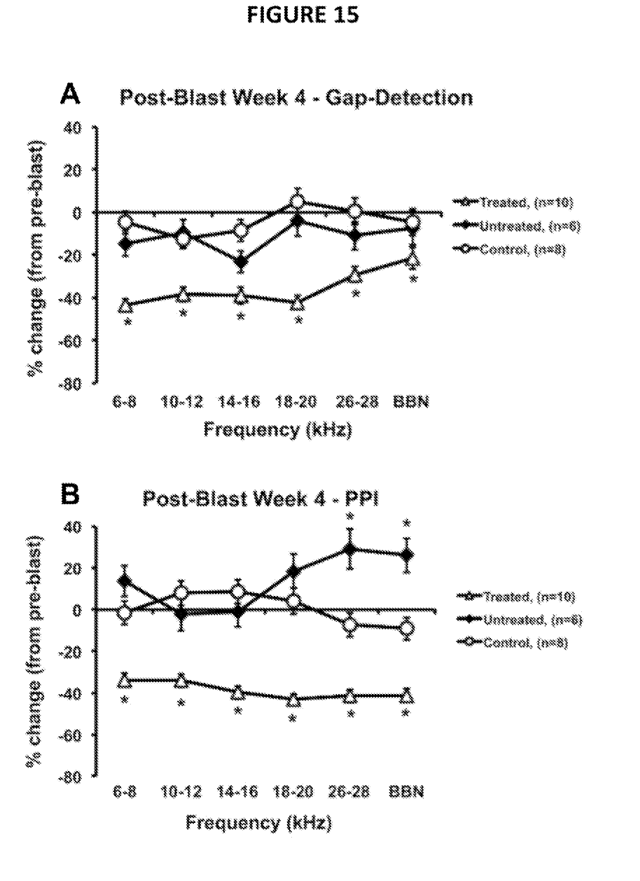

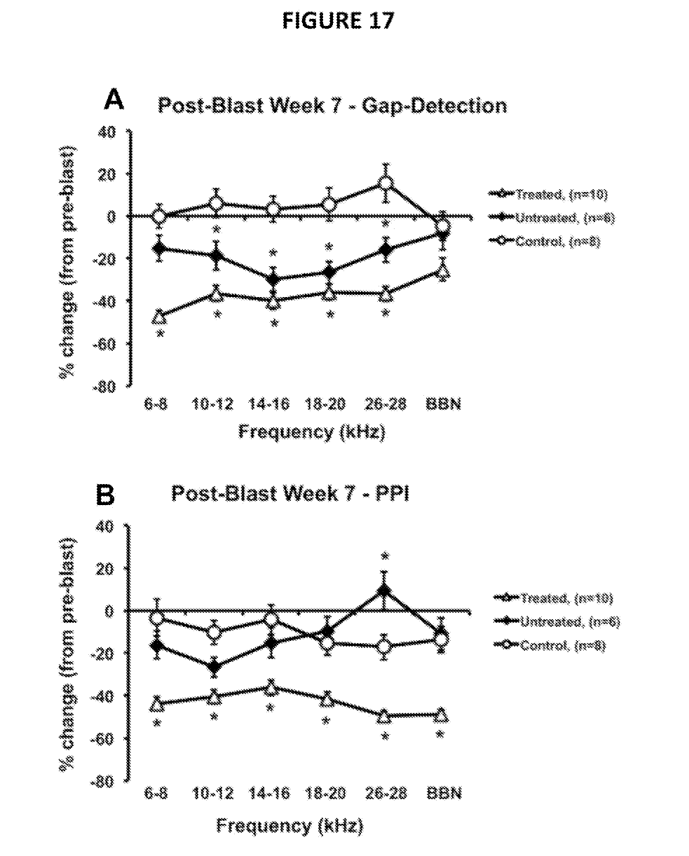

Compared to the Treated group, the Untreated group showed significantly less reduction in startle force and on occasion displayed increased startle force. The exception was during the first week post-blast, where the Untreated group sustained startle force reduction during all background noises (FIG. 13A) and without background noise (FIG. 13B). Following 1 week post-blast, they only displayed occasional reductions in startle force compared to the Sham group, including during 14-16 kHz at 3 weeks post-blast (FIG. 14A) and at 6 weeks post-blast (FIG. 16A), but showed greater startle force reduction at 7 weeks post-blast during 10-28 kHz background noise (FIG. 17A). The Untreated group also demonstrated increased startle force without background noise compared to the Sham group at 3 weeks post-blast near 6-8 kHz (FIG. 14B), at 4 weeks post-blast near 26-28 kHz and BBN (FIG. 15B), at 6 weeks post-blast near all prepulse conditions (FIG. 16B), which was mostly due to reduction in the Sham group startle force, and at 7 weeks post-blast near 26-28 kHz prepulses (FIG. 17B). The lack of overall startle force decrease seen in the Untreated group, compared to the Treated group, as well as the occasional increases in startle force may indicate increased startle responsivity. This could in turn implicate the presence of hyperacusis-like precepts and behavior in the Untreated group.

TABLE 3
Statistical analysis - Startle-only startle force change during background noise
GAP STARTLE FORCE

| | 6-8 kHz | 10-12 kHz | 14-16 kHz | 18-20 kHz | 26-28 kHz | BBN |
|---|---|---|---|---|---|---|
| PB1Wk | F_{(2,489)} = | F_{(2,492)} = | F_{(2,488)} = | F_{(2,494)} = | F_{(2,492)} = | F_{(2,494)} = |
| | 51.021 | 48.517 | 43.110 | 61.021 | 44.272 | 37.198 |
| | T-U *T-S | T-U *T-S | T-U *T-S | T-U *T-S | T-U *T-S | T-U *T-S |
| | *U-S | *U-S | *U-S | *U-S | *U-S | *U-S |
| PB3Wk | F_{(2,541)} = | F_{(2,543)} = | F_{(2,537)} = | F_{(2,540)} = | F_{(2,536)} = | F_{(2,536)} = |
| | 23.587 | 10.955 | 17.614 | 15.177 | 5.101 | 7.156 |
| | *T-U *T-S | *T-U *T-S | *T-U *T-S | *T-U *T-S | T-U *T-S | *T-U *T-S |
| | U-S | U-S | *U-S | U-S | U-S | U-S |
| PB4Wk | F_{(2,479)} = | F_{(2,473)} = | F_{(2,476)} = | F_{(2,478)} = | F_{(2,479)} = | F_{(2,471)} = |
| | 22.028 | 12.918 | 11.817 | 25.314 | 8.465 | 2.347 |
| | T-U *T-S | T-U *T-S | T-U *T-S | T-U *T-S | T-U *T-S | T-U *T-S |
| | *U-S | *U-S | *U-S | *U-S | *U-S | *U-S |
| PB6Wk | F_{(2,470)} = | F_{(2,468)} = | F_{(2,468)} = | F_{(2,471)} = | F_{(2,465)} = | F_{(2,471)} = |
| | 7.715 | 7.875 | 11.199 | 19.205 | 10.786 | 0.398 |
| | T-U *T-S | T-U *T-S | T-U *T-S | *T-U *T-S | *T-U *T-S | T-U *T-S |
| | U-S | U-S | *U-S | U-S | U-S | U-S |
| PB7Wk | F_{(2,518)} = | F_{(2,514)} = | F_{(2,514)} = | F_{(2,511)} = | F_{(2,511)} = | F_{(2,510)} = |
| | 27.663 | 15.356 | 20.617 | 14.425 | 17.359 | 3.066 |
| | *T-U *T-S | T-U *T-S | T-U *T-S | T-U *T-S | T-U *T-S | T-U T-S |
| | U-S | *U-S | *U-S | *U-S | *U-S | U-S |

TABLE 4
Statistical analysis - Startle only startle force change without background noise
PPI STARTLE FORCE

| | 6-8 kHz | 10-12 kHz | 14-16 kHz | 18-20 kHz | 26-28 kHz | BBN |
|---|---|---|---|---|---|---|
| PB1Wk | F_{(2,487)} = | F_{(2,493)} = | F_{(2,492)} = | F_{(2,487)} = | F_{(2,485)} = | F_{(2,484)} = |
| | 20.054 | 46.724 | 42.382 | 33.683 | 44.662 | 38.573 |
| | T-U *T-S | T-U *T-S | T-U *T-S | T-U *T-S | T-U *T-S | T-U *T-S |
| | *U-S | *U-S | *U-S | *U-S | *U-S | *U-S |
| PB3Wk | F_{(2,400)} = | F_{(2,411)} = | F_{(2,403)} = | F_{(2,409)} = | F_{(2,410)} = | F_{(2,401)} = |
| | 11.114 | 18.546 | 15.392 | 11.519 | 11.443 | 22.987 |
| | *T-U T-S | *T-U *T-S | *T-U *T-S | *T-U *T-S | *T-U *T-S | *T-U *T-S |
| | *U-S | U-S | U-S | U-S | U-S | *U-S |
| PB4Wk | F_{(2,541)} = | F_{(2,537)} = | F_{(2,529)} = | F_{(2,534)} = | F_{(2,537)} = | F_{(2,537)} = |
| | 23.884 | 19.044 | 29.029 | 36.415 | 37.258 | 41.300 |
| | *T-U *T-S | *T-U *T-S | *T-U *T-S | *T-U *T-S | *T-U *T-S | *T-U *T-S |
| | U-S | U-S | U-S | U-S | *U-S | *U-S |
| PB6Wk | F_{(2,474)} = | F_{(2,466)} = | F_{(2,473)} = | F_{(2,465)} = | F_{(2,469)} = | F_{(2,468)} = |
| | 6.584 | 15.714 | 14.496 | 20.574 | 7.656 | 17.749 |
| | *T-U T-S | *T-U T-S | *T-U T-S | *T-U T-S | *T-U T-S | *T-U T-S |
| | *U-S | *U-S | *U-S | *U-S | *U-S | *U-S |
| PB7Wk | F_{(2,549)} = | F_{(2,547)} = | F_{(2,559)} = | F_{(2,547)} = | F_{(2,547)} = | F_{(2,544)} = |
| | 12.547 | 12.161 | 9.265 | 12.461 | 28.043 | 18.249 |
| | *T-U *T-S | T-U *T-S | *T-U *T-S | *T-U *T-S | *T-U *T-S | *T-U *T-S |
| | U-S | U-S | U-S | U-S | *U-S | U-S |

D. ABR Threshold Shifts.

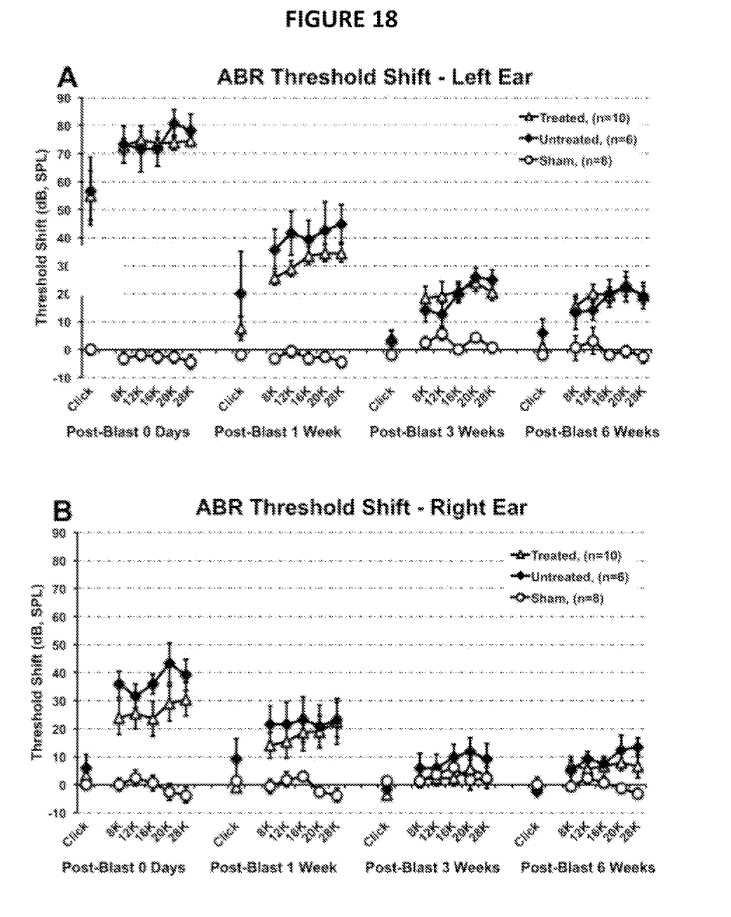

ABR recordings were conducted in the left (unplugged) and right (plugged) ears to determine if sildenafil had any therapeutic effects on blast-induced threshold shifts. Although unilateral blast exposure by occluding the right ear with an earplug was attempted, the right ears still incurred threshold shifts of over 40 dB (FIG. 18). In addition, while there were no significant differences in threshold shifts between the Treated and Untreated groups at individual frequencies, an overall reduction in threshold shifts for the Treated group in the right ear at post-blast day 0 (F_{(2,117)}=69.306, p=0.005; FIG. 18B) and in the left ear at post-blast week 1 (F_{(2,117)}=150.927, p=0.002; FIG. 18A) was observed, suggesting that sildenafil reduced hearing impairment.

At post-blast day 0, the Treated and Untreated groups showed significant threshold shifts in the left and right ears (statistics in Tables 5 and 6, respectively) compared to the Sham group. The Treated group exhibited significant left ear threshold shifts at click and 8-28 kHz, and in the right ear from 8-28 kHz. Untreated rats also exhibited threshold shifts in the left ear at click and 8-28 kHz, and in the right ear from 8-28 kHz.

By 1 week post-blast, significant threshold shifts remained in the left ear at all frequencies and in the right ear at higher frequencies. The Treated group showed threshold shifts in the left ear from 8-28 kHz, and in the right ear at 20 and 28 kHz. The Untreated group showed threshold shifts in the left ear from 8-28 kHz, and in the right ear at 20 and 28 kHz.

At 3 weeks post-blast, threshold shifts largely recovered in the right ear, but remained at the higher frequencies in the left ear. The Treated group displayed significant threshold shifts at 8 and 16-28 kHz, while the Untreated group showed threshold shifts from 16-28 kHz.

Threshold shifts remained relatively stable from 3-6 weeks post-blast. During post-blast week 6, the Treated group exhibited left ear threshold shifts from 8-28 kHz and the Untreated group exhibited shifts from 16-28 kHz.

TABLE 5
Statistical analysis - ABR threshold shifts in the left ear
ABR THRESHOLD SHIFTS - LEFT EAR

| | 8 kHz | 12 kHz | 16 kHz | 20 kHz | 28 kHz | Click |
|---|---|---|---|---|---|---|
| PB0D | F_{(2,21)} = | F_{(2,21)} = | F_{(2,21)} = | F_{(2,21)} = | F_{(2,21)} = | F_{(2,21)} = |
| | 139.996 | 108.523 | 177.006 | 208.218 | 207.599 | 15.103 |
| | T-U *T-S | T-U *T-S | T-U *T-S | T-U *T-S | T-U *T-S | T-U *T-S |
| | *U-S | *U-S | *U-S | *U-S | *U-S | *U-S |
| PB1Wk | F_{(2,21)} = | F_{(2,21)} = | F_{(2,21)} = | F_{(2,21)} = | F_{(2,21)} = | F_{(2,21)} = |
| | 29.823 | 25.256 | 32.691 | 22.008 | 42.075 | 2.012 |
| | T-U *T-S | T-U *T-S | T-U *T-S | T-U *T-S | T-U *T-S | T-U *T-S |
| | *U-S | *U-S | *U-S | *U-S | *U-S | *U-S |
| PB3Wk | F_{(2,21)} = | F_{(2,21)} = | F_{(2,21)} = | F_{(2,21)} = | F_{(2,21)} = | F_{(2,21)} = |
| | 5.476 | 2.574 | 17.257 | 16.756 | 20.135 | 1.525 |
| | T-U *T-S | T-U T-S | T-U *T-S | T-U *T-S | T-U *T-S | T-U T-S |
| | U-S | U-S | *U-S | *U-S | *U-S | U-S |
| PB6Wk | F_{(2,21)} = | F_{(2,21)} = | F_{(2,21)} = | F_{(2,21)} = | F_{(2,21)} = | F_{(2,21)} = |
| | 3.834 | 5.127 | 17.589 | 13.294 | 14.890 | 2.652 |
| | T-U *T-S | T-U *T-S | T-U *T-S | T-U *T-S | T-U *T-S | T-U T-S |
| | U-S | U-S | *U-S | *U-S | *U-S | U-S |

TABLE 6
Statistical analysis - ABR threshold shifts in the right ear
ABR THRESHOLD SHIFTS - RIGHT EAR

| | 8 kHz | 12 kHz | 16 kHz | 20 kHz | 28 kHz | Click |
|---|---|---|---|---|---|---|
| PB0D | F_{(2,21)} = | F_{(2,21)} = | F_{(2,21)} = | F_{(2,21)} = | F_{(2,21)} = | F_{(2,21)} = |
| | 11.696 | 9.980 | 11.119 | 15.103 | 17.59 | 0.782 |
| | T-U *T-S | T-U *T-S | T-U *T-S | T-U *T-S | T-U *T-S | T-U T-S |
| | *U-S | *U-S | *U-S | *U-S | *U-S | U-S |
| PB1Wk | F_{(2,21)} = | F_{(2,21)} = | F_{(2,21)} = | F_{(2,21)} = | F_{(2,21)} = | F_{(2,21)} = |
| | 5.637 | 2.853 | 3.006 | 5.844 | 5.398 | 2.430 |
| | T-U T-S | T-U T-S | T-U T-S | T-U T-S | T-U *T-S | T-U T-S |
| | *U-S | U-S | U-S | U-S | *U-S | U-S |
| PB3Wk | F_{(2,21)} = | F_{(2,21)} = | F_{(2,21)} = | F_{(2,21)} = | F_{(2,21)} = | F_{(2,21)} = |
| | 0.443 | 0.253 | 1.052 | 1.309 | 0.713 | 8.076 |
| | T-U T-S | T-U T-S | T-U T-S | T-U T-S | T-U T-S | T-U *T-S |
| | U-S | U-S | U-S | U-S | U-S | U-S |
| PB6Wk | F_{(2,21)} = | F_{(2,21)} = | F_{(2,21)} = | F_{(2,21)} = | F_{(2,21)} = | F_{(2,21)} = |
| | 2.004 | 1.273 | 1.578 | 4.270 | 4.724 | 0.501 |
| | T-U T-S | T-U T-S | T-U T-S | T-U T-S | T-U T-S | T-U T-S |
| | U-S | U-S | U-S | U-S | U-S | U-S |

### IV. Discussion

A. Therapeutic Effect of Sildenafil on Tinnitus and Hearing Loss.

Although sildenafil treatment did not prevent immediate tinnitus onset, it suppressed high-frequency tinnitus from 3-6 weeks post-blast, after which high-frequency tinnitus reemerged. Broadband tinnitus followed by high-frequency tinnitus was consistent with previous reports, except that tinnitus was transient, most likely due to decreased blast exposure parameters (single blast, 14 psi) that were used. Although blast exposure resulted in both strong startle force reduction and hearing loss during the first week post-blast, startle force remained significantly higher than the noise floor (data not shown) and several studies have reported tinnitus onset following acoustic trauma, supporting the currently disclosed post-blast week 1 tinnitus findings. As a whole, the currently disclosed results suggest sildenafil has the capacity to suppress tinnitus, but this occurs in a time-dependent fashion. Several factors may explain why sildenafil was effective at treating high-frequency tinnitus several weeks after blast exposure.

First, and without being bound by theory, immediately following blast, tinnitus-inducing damage to the auditory system may have passed a therapeutic threshold. This theory is supported by the recovery progression of hearing impairment and the correlation between hearing impairment and tinnitus severity. Although sildenafil reduced overall hearing threshold shifts in the right (plugged) ear at post-blast day 0 and in the left (unplugged) ear at post-blast week 1, it did not significantly reduce left ear threshold shifts at post-blast day 0. The threshold shifts sustained from the latter ear and time point were in the range of 65-80 dB and much higher than the other threshold shifts. This suggests that while sildenafil offers some protection against hearing impairment, it is ineffective past a certain amount of damage. The fact that the left ears of both groups sustained comparable threshold shifts indicates a similar degree of injury. Unilateral noise exposure and unilateral temporary threshold shifts alone are adequate to induce tinnitus and impair Gap-detection. In the currently described studies, strong tinnitus driven primarily by damage to the left ear may have negated the effect of any reduction in hearing impairment on behavioral performance. In addition, it is possible that sildenafil can only suppress tinnitus once the auditory system has recovered to a certain degree following trauma. Indeed, sildenafil only began suppressing tinnitus at 3 weeks post-blast, by which time there were no longer any threshold shift differences between Treated and Untreated rats.

Also without being bound by theory, another reason why sildenafil suppressed tinnitus from post-blast week 3-6 but not week 1 may be that acute and more chronic forms of tinnitus have different generators, and these chronic generators are more susceptible to sildenafil treatment. Very little is known about the differences between acute versus chronic generators of tinnitus, in part due to the difficulty of separating acute tinnitus generators from hearing loss and simultaneously confirming tinnitus perception with behavioral testing. Some evidence has suggested that the dorsal cochlear nucleus and inferior colliculus undergo an initial decrease in spontaneous activity immediately after noise exposure, and this hypoactivity transitions to hyperactivity within days to months afterwards (Kaltenbach, J. A., et al., Hear Res 147:282-292, 2000; Wang, H. et al., Hear Res 279:111-117, 2011). A clinical study that used vardenafil to treat chronic tinnitus, however, found no therapeutic effect (Mazurek, B. et al., J Negat Results Biomed 8: 3, 2009), and attributed this to not administering vardenafil sooner after tinnitus-inducing trauma. Therefore, while treating rats with sildenafil immediately after blast exposure did not prevent acute tinnitus, it may have prevented the plastic changes responsible for longer-lasting tinnitus. These plastic changes may have been further modulated by sildenafil treatment during the third week post-blast, but were allowed to take place by post-blast week 7 when high-frequency tinnitus reemerged.

Little research has been conducted on the effect of PDE-5 inhibitors like sildenafil and vardenafil on tinnitus (Mazurek, B. et al., J Negat Results Biomed 8: 3, 2009). However, studies have found that they can yield hearing protection and faster hearing recovery from acoustic trauma (Jaumann, M., et al., Nat Med 18:252-259, 2012; Zhang, S. J. et al., Chinese J of Otorhinolaryngology Head and Neck Surgery 46:844-847, 2011), as well as adverse effects such as onset hearing impairment (Mukherjee, B. and Shivakumar, T., J Laryngol Otol 121:395-397, 2007; Hong, B. N. et al., Biological & Pharmaceutical Bulletin 31:1981-1984, 2008; Maddox, P. T. et al., Laryngoscope 119:1586-1589 2009; Okuyucu, S. et al., J Laryngol Otol 123:718-722, 2009; McGwin, G. Jr. et al., Arch Otolaryngol Head Neck Surg 136:488-492, 2010; Snodgrass, A. J. et al., Pharmacotherapy 30:112, 2010; Khan, A. S. et al., Laryngoscope 121:1049-1054, 2011; Thakur, J. S. et al., Laryngoscope 123:1527-1530, 2013), though the latter can be transient. On the contrary, other findings suggest that PDE-5 inhibitors yield no effect on hearing (Mazurek, B. et al., J Negat Results Biomed 8: 3, 2009; Giuliano, F. et al., Int J of Clin Practice 64:240-255, 2010.; Thakur, J. S. et al., Laryngoscope 123:1527-1530, 2013). It may be that PDE-5 inhibitors can have negative effects from long-term usage and/or high dosage, but that when administered directly before or after acoustic trauma at a low dosage, they can provide therapeutic effects. PDE-5 inhibitors have been shown to enhance the NO-cGMP pathway, which in turn leads to increased vasodilation and blood flow. Intense noise exposure has been shown to reduce partial oxygen pressure and cochlear blood flow Scheibe, F. et al., Hear Res 63:19-25, 1992; Scheibe, F. et al., Eur Arch Otorhinolaryngol 250:281-285, 1993; Lamm, K. et al., Ann N Y Acad Sci 884:233-248, 1999), which can at extremes lead to near total degeneration of the inner ear (Ren, T. et al., Hear Res 92:30-37, 1995; Otake, H. et al., Acta Otolaryngol 129:127-131, 2009). Therefore, and without being bound by theory, initial reduction of hearing impairment and later suppression of high-frequency tinnitus in the Treated group may be related to improved blood flow to the cochlea and peripheral auditory system. Without being bound by theory, tinnitus suppression and reduced hearing impairment may have also been due to protective effects from the Akt (Protein Kinase B) pathway and endothelial nitric oxide synthase (eNOS) activation via sildenafil administration. Sildenafil has been shown to activate the Akt pathway, which can enhance neurogenesis following stroke (Wang, L. et al., J Cereb Blood Flow Metab 25(9): 1150-1158, 2005) and inhibit apoptotic signals, resulting in improved neuronal cell survival and functional recovery following controlled-cortical impact TBI (Noshita, N. et al., Neurobiol Dis 9(3): 294-304, 2002; Wu, Y. et al., J Biomed Biotechnol 2011: 384627, 2011). An immunohistochemistry study has revealed strong staining of phosphorylated-Akt inside and underneath inner hair cells (Hess A., et al., Eur Arch Otorhinolaryngol 263:75-78, 2006). At the same time, sildenafil and Akt can increase production of eNOS (Das, A. et al., J Biol Chem 280(13): 12944-12955, 2005; Yuan, Z. et al., Invest Ophthalmol Vis Sci 49(2): 720-725, 2008; Shao, Z. H. et al., J Cell Biochem 107(4): 697-705, 2009; Mammi, C. et al., PLoS One 6(1): e14542, 2011), which has been found in the cochlear microvasculature and spiral ganglia (Gosepath, Brain Res. 747(1):26-33, 1997; Franz, 1996) and has been shown to maintain cerebral blood flow and blood pressure following controlled cortical impact TBI (Lundblad, C. et al., J Neurotrauma 26(11): 1953-1962, 2009) and counteract oxidative stress (Chiueh, Ann N Y Aced Sci. 890:301-11, 1999).

B. Therapeutic Effect of Sildenafil on TBI and Hyperacusis-Like Behavior.

While both the Treated and Untreated groups exhibited an initial decrease in startle force during the startle only condition, the Treated group demonstrated a long-lasting decrease across almost all conditions from the third week onward. In contrast, the Untreated group showed anywhere between little change to occasional decreases and increases in startle force. At the same time, the Sham group demonstrated decreased startle force at 1 week post-blast and without background noise at post-blast week 6, although these were isolated instances and most likely reflect natural variability in startle force (i.e. transportation between buildings following pseudo-blast).

Previous studies have shown that both noise exposure and hearing loss (Longenecker, R. J. and Galazyuk, A. V., J Assoc Res Otolaryngol 12:647-658, 2011; Rybalko, N., et al., Physiology & Behavior 102:453-458, 2011; Lobarinas, E. et al., Acta Otolaryngol Suppl 13-19, 2006), as well as fluid percussion TBI (Wiley, J. L. et al., Brain Res 716:47-52, 1996; Lu, J., et al., J Neuropharmacology 44:253-263, 2003) can reduce acoustic startle force in animals. Although the auditory impact from blast exposure can potentially reduce startle force, this may not be the case in the currently described studies because lasting hearing threshold shifts only occurred at 16-28 kHz in the left ear. Others have indicated that acoustic trauma and hearing loss that primarily affect higher frequency ranges can result in little change to an actual increase in startle force, the latter of which may be due to overrepresentation of lower frequencies and linked to hyperacusis (Ison, J. R. and Allen, P. D., J of the Association for Research in Otolaryngology: JARO 4:495-504, 2003; Ison, J. R. et al., J Assoc Res Otolaryngol 8:539-550, 2007; Sun, W. et al., Neuroscience 159:325-334, 2009; Lu, J. et al., Neuroscience 189:187-198, 2011; Sun, W. et al., Brain Res 1485:108-116, 2012; Chen, G. et al., J Assoc Res Otolaryngol 14:413-424, 2013; Pace, E. and Zhang, J. S., PLoS ONE 8:e75011, 2013). Therefore, blast-induced TBI may be a contributing factor to the reduced startle force observed in the Treated group. In humans, blast has been associated with orbitofrontal damage (Mac Donald, C. L., et al., N Engl J Med 364:2091-2100, 2011), which has been linked to reduced startle force (Angrilli, A. et al., Neuropsychologia 46:1179-1184, 2008). Though blast-induced TBI was not measured, findings from studies suggesting blast-induced neuroplasticity (Mao, J. C. et al., J Neurotrauma 29(2): 430-444, 2012) and other studies using similar blasting parameters (1-3 blasts, <22 psi), including acute mitochondrial dysfunction (Arun, P. et al., J Neurotrauma 30(19):1645-51, 2013), dysregulation in cholinergic and inflammatory pathway-related genes (Valiyaveettil, M. et al., J Rehabil Res Dev 49:1153-1162, 2012), and disruption of the blood brain barrier (Abdul-Muneer, P. M. et al., Free Radical Biology & Medicine 60:282-291, 2013), all suggest the likely induction of TBI in the currently described studies. Furthermore, blasted rats demonstrated a dose-dependent increase in surface righting latency, which has been correlated with TBI severity (Li, Y. et al., Stapp Car Crash J 55:25-47, 2011; Li, Y. et al., J Neurotrauma 28:1767-1782, 2011). Although blast injury is known to cause vestibular deficits, including dizziness, disequilibrium, and compromised spatial perception and navigation (Franke L. M. et al., J Rehabil Res Dev 49:985-994, 2012), rats in the currently described studies remained completely unconscious prior to regaining consciousness and righting themselves, suggesting that vestibular defects had limited influence on surface righting latency.

### V. Conclusions

Sildenafil treatment reduced behavioral evidence of high-frequency tinnitus as well as hearing loss and hyperacusis-like behavior. Without being bound by theory, these therapeutic effects are likely due to vasodilation and improved blood flow to the peripheral and central auditory system, which disrupted the normative and potentially maladaptive plastic changes following blast-induced trauma. In spite of these effects, sildenafil treatment appeared to be limited to a certain degree of tinnitus- and hearing-related damage, which may have been caused by blast-induced TBI. Taken together, our results suggest that sildenafil can help attenuate adverse auditory consequences of blast exposure. Increasing sildenafil dosage, as well as administering sildenafil prior to blast exposure, may be promising treatment routes, in addition to combination with other approaches such as electrical stimulation and sound therapy to optimally modulate the underlying pathological neuroplasticity. Given the considerable physical and psychological detriments associated with blast-induced tinnitus, hearing loss, and TBI, there is an urgent need for clearer understanding of the overall problem and improved treatment outcomes.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transitional phrase “consisting essentially of” limits the scope of the embodiment to the specified elements, steps, ingredients or components and to those that do not materially affect the embodiment. As used herein, a material effect would cause a statistically significant reduction in a PDE-I(s)′ability to improve Gap-detection, PPI performance, and/or ABR following blast exposure in an animal model described herein.

Unless otherwise indicated, all numbers used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to publications, patents and/or patent applications (collectively “references”) throughout this specification. Each of the cited references is individually incorporated herein by reference for their particular cited teachings.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of ordinary skill in the art, such as the Oxford Dictionary of Biochemistry and Molecular Biology (Ed. Anthony Smith, Oxford University Press, Oxford, 2004).

1. A method of treating high-frequency blast-induced tinnitus in a subject in need thereof comprising administering to the subject a therapeutically effective amount of a phosphodiesterase inhibitor (PDE-I) within a time period associated with exposure to a blast thereby providing a therapeutic treatment by reducing in the subject 26-28 kHz blast-induced tinnitus 3-6 weeks following exposure to the blast.

2. A method of claim 1 where the PDE-I is a PDE-5 inhibitor.

3. A method of claim 2 wherein the PDE-I is sildenafil.

4. A method of claim 1 wherein the time period is within 24 hours of exposure to a blast; within 1 hour of exposure to a blast; within 10 minutes of exposure to a blast; or within 5 minutes of exposure to a blast.

5. A method of claim 1 wherein the blast creates a pressure wave of 10 psi or greater; 20 psi or greater; or 22 psi or greater.

6. A method of claim 1 wherein the therapeutic treatment reduces 26-28 kHz blast-induced tinnitus in the subject 3 weeks following exposure to the blast.

7. A method of claim 1 wherein the therapeutic treatment reduces 26-28 kHz blast-induced tinnitus in the subject 4 weeks following exposure to the blast.

8. A method of claim 1 wherein the therapeutic treatment reduces 26-28 kHz blast-induced tinnitus in the subject 5 weeks following exposure to the blast.

9. A method of claim 1 wherein the therapeutic treatment reduces 26-28 kHz blast-induced tinnitus in the subject 6 weeks following exposure to the blast.

10. A method of treating high-frequency blast-induced tinnitus in a subject in need thereof comprising administering to the subject a therapeutically effective amount of a phosphodiesterase inhibitor (PDE-I) within a time period associated with exposure to a blast thereby providing a therapeutic treatment by reducing in the subject 18-20 kHz and/or 26-28 kHz blast-induced tinnitus 3-4 weeks following exposure to the blast.

11. A method of claim 10 where the PDE-I is a PDE-5 inhibitor.

12. A method of claim 11 wherein the PDE-I is sildenafil.

13. A method of claim 10 wherein the time period is within 24 hours of exposure to a blast; within 10 hour of exposure to a blast; within 10 minutes of exposure to a blast; or within 5 minutes of exposure to a blast.

14. A method of claim 10 wherein the blast creates a pressure wave of 10 psi or greater; 20 psi or greater; or 22 psi or greater.

15. A method of claim 10 wherein the therapeutic treatment reduces 18-20 kHz blast-induced tinnitus in the subject 3 weeks following exposure to the blast.

16. A method of claim 10 wherein the therapeutic treatment reduces 18-20 kHz blast-induced tinnitus in the subject 4 weeks following exposure to the blast.

17. A method of claim 10 wherein the therapeutic treatment reduces 26-28 kHz blast-induced tinnitus in the subject 3 weeks following exposure to the blast.

18. A method of claim 10 wherein the therapeutic treatment reduces 26-28 kHz blast-induced tinnitus in the subject 4 weeks following exposure to the blast.

19. A method of claim 10 wherein the therapeutic treatment reduces 18-20 kHz and 26-28 kHz blast-induced tinnitus in the subject 3-4 weeks following exposure to the blast.

20. A method of treating hearing loss in a subject in need thereof comprising administering to the subject a therapeutically effective amount of a phosphodiesterase inhibitor (PDE-I) within a time period associated with exposure to a blast thereby providing a therapeutic treatment by reducing in the subject 6-8 kHz hearing loss 0-2 weeks following exposure to the blast; 14-16 kHz hearing loss 0-4 weeks following exposure to the blast and/or 18-20 kHz hearing loss 0-2 weeks and/or 4-6 weeks following exposure to the blast.

21. A method of claim 20 where the PDE-I is a PDE-5 inhibitor.

22. A method of claim 21 wherein the PDE-I is sildenafil.

23. A method of claim 20 wherein the time period is within 24 hours of exposure to a blast; within 1 hour of exposure to a blast; within 10 minutes of exposure to a blast; or within 5 minutes of exposure to a blast.

24. A method of claim 20 wherein the blast creates a pressure wave of 10 psi or greater; 20 psi or greater; or 22 psi or greater.

25. A method of claim 20 wherein the therapeutic treatment reduces 6-8 kHz hearing loss in the subject 0 weeks following exposure to the blast.

26. A method of claim 20 wherein the therapeutic treatment reduces 6-8 kHz hearing loss in the subject 1 week following exposure to the blast.

27. A method of claim 20 wherein the therapeutic treatment reduces 6-8 kHz hearing loss in the subject 2 weeks following exposure to the blast.

28. A method of claim 20 wherein the therapeutic treatment reduces 14-16 kHz hearing loss in the subject 0 weeks following exposure to the blast.

29. A method of claim 20 wherein the therapeutic treatment reduces 14-16 kHz hearing loss in the subject 1 week following exposure to the blast.

30. A method of claim 20 wherein the therapeutic treatment reduces 14-16 kHz hearing loss in the subject 2 weeks following exposure to the blast.

31. A method of claim 20 wherein the therapeutic treatment reduces 14-16 kHz hearing loss in the subject 3 weeks following exposure to the blast.

32. A method of claim 20 wherein the therapeutic treatment reduces 14-16 kHz hearing loss in the subject 4 weeks following exposure to the blast.

33. A method of claim 20 wherein the therapeutic treatment reduces 18-20 kHz hearing loss in the subject 0 weeks following exposure to the blast.

34. A method of claim 20 wherein the therapeutic treatment reduces 18-20 kHz hearing loss in the subject 1 week following exposure to the blast.

35. A method of claim 20 wherein the therapeutic treatment reduces 18-20 kHz hearing loss in the subject 2 weeks following exposure to the blast.

36. A method of claim 20 wherein the therapeutic treatment reduces 18-20 kHz hearing loss in the subject 4 weeks following exposure to the blast.

37. A method of claim 20 wherein the therapeutic treatment reduces 18-20 kHz hearing loss in the subject 5 weeks following exposure to the blast.

38. A method of claim 20 wherein the therapeutic treatment reduces 18-20 kHz hearing loss in the subject 6 weeks following exposure to the blast.

39. A method of treating high-frequency blast-induced tinnitus and hearing loss in a subject in need thereof comprising administering to the subject a therapeutically effective amount of a phosphodiesterase inhibitor (PDE-I) within a time period associated with exposure to a blast thereby providing a therapeutic treatment by reducing in the subject (i) 26-28 kHz blast-induced tinnitus 3-6 weeks following exposure to the blast (ii) 18-20 kHz and/or 26-28 kHz blast-induced tinnitus 3-4 weeks following exposure to the blast; and (iii) 6-8 kHz hearing loss 0-2 weeks following exposure to the blast; 14-16 kHz hearing loss 0-4 weeks following exposure to the blast and/or 18-20 kHz hearing loss 0-2 weeks and/or 4-6 weeks following exposure to the blast.

40. A method of claim 39 where the PDE-I is a PDE-5 inhibitor.

41. A method of claim 40 wherein the PDE-I is sildenafil.

42. A method of claim 39 wherein the time period is within 24 hours of exposure to a blast; within 1 hour of exposure to a blast; within 10 minutes of exposure to a blast; or within 5 minutes of exposure to a blast.

43. A method of claim 39 wherein the blast creates a pressure wave of 10 psi or greater; 20 psi or greater; or 22 psi or greater.

44. A method of claim 39 wherein the therapeutic treatment reduces 26-28 kHz blast-induced tinnitus in the subject 3 weeks following exposure to the blast.

45. A method of claim 39 wherein the therapeutic treatment reduces 26-28 kHz blast-induced tinnitus in the subject 4 weeks following exposure to the blast.

46. A method of claim 39 wherein the therapeutic treatment reduces 26-28 kHz blast-induced tinnitus in the subject 5 weeks following exposure to the blast.

47. A method of claim 39 wherein the therapeutic treatment reduces 26-28 kHz blast-induced tinnitus in the subject 6 weeks following exposure to the blast.

48. A method of claim 39 wherein the therapeutic treatment reduces 18-20 kHz blast-induced tinnitus in the subject 3 weeks following exposure to the blast.

49. A method of claim 39 wherein the therapeutic treatment reduces 18-20 kHz blast-induced tinnitus in the subject 4 weeks following exposure to the blast.

50. A method of claim 39 wherein the therapeutic treatment reduces 26-28 kHz blast-induced tinnitus in the subject 3 weeks following exposure to the blast.

51. A method of claim 39 wherein the therapeutic treatment reduces 26-28 kHz blast-induced tinnitus in the subject 4 weeks following exposure to the blast.

52. A method of claim 39 wherein the therapeutic treatment reduces 18-20 kHz and 26-28 kHz blast-induced tinnitus in the subject 3-4 weeks following exposure to the blast.

53. A method of claim 39 wherein the therapeutic treatment reduces 6-8 kHz hearing loss in the subject 0 weeks following exposure to the blast.

54. A method of claim 39 wherein the therapeutic treatment reduces 6-8 kHz hearing loss in the subject 1 week following exposure to the blast.

55. A method of claim 39 wherein the therapeutic treatment reduces 6-8 kHz hearing loss in the subject 2 weeks following exposure to the blast.

56. A method of claim 39 wherein the therapeutic treatment reduces 14-16 kHz hearing loss in the subject 0 weeks following exposure to the blast.

57. A method of claim 39 wherein the therapeutic treatment reduces 14-16 kHz hearing loss in the subject 1 week following exposure to the blast.

58. A method of claim 39 wherein the therapeutic treatment reduces 14-16 kHz hearing loss in the subject 2 weeks following exposure to the blast.

59. A method of claim 39 wherein the therapeutic treatment reduces 14-16 kHz hearing loss in the subject 3 weeks following exposure to the blast.

60. A method of claim 39 wherein the therapeutic treatment reduces 14-16 kHz hearing loss in the subject 4 weeks following exposure to the blast.

61. A method of claim 39 wherein the therapeutic treatment reduces 18-20 kHz hearing loss in the subject 0 weeks following exposure to the blast.

62. A method of claim 39 wherein the therapeutic treatment reduces 18-20 kHz hearing loss in the subject 1 week following exposure to the blast.

63. A method of claim 39 wherein the therapeutic treatment reduces 18-20 kHz hearing loss in the subject 2 weeks following exposure to the blast.

64. A method of claim 39 wherein the therapeutic treatment reduces 18-20 kHz hearing loss in the subject 4 weeks following exposure to the blast.

65. A method of claim 39 wherein the therapeutic treatment reduces 18-20 kHz hearing loss in the subject 5 weeks following exposure to the blast.

66. A method of claim 39 wherein the therapeutic treatment reduces 18-20 kHz hearing loss in the subject 6 weeks following exposure to the blast.
